(19) United States
(12) Patent Application Publication
(10) Pub. No.: US 2007/0026450 A1
(43) Pub. Date: Feb. 1, 2007

(54) METHODS FOR THE TREATMENT OF CARCINOMA

(76) Inventors: Mary E. Gerritsen, San Mateo, CA (US); Franklin V. Peale JR., San Carlos, CA (US); Thomas D. Wu, San Francisco, CA (US)

Correspondence Address: GENENTECH, INC., 1 DNA WAY, SOUTH SAN FRANCISCO, CA, 94080, US

(21) Appl. No.: 11/535,366

(22) Filed: Sep. 26, 2006

Related U.S. Application Data
(63) Continuation of application No. 10/271,690, filed on Oct. 16, 2002.
(60) Provisional application No. 60/344,534, filed on Oct. 18, 2001.

(51) Int. Cl. C12Q 1/68; G01N 33/574; C12P 21/06; C12N 5/06; C07K 16/40; C07K 16/30

(52) U.S. Cl. 435/6; 435/7.23; 435/69.1; 435/326; 435/320.1; 530/388.8; 530/388.26

(57) ABSTRACT

The invention concerns compositions and methods for the diagnosis and treatment of neoplastic cell growth and proliferation in mammals, including humans. The invention is based upon the identification of genes that are amplified in the genome of tumor cells, such as renal cell carcinoma. Such gene amplification is expected to be associated with the overexpression of the gene product as compared to normal cells of the same tissue type and contribute to tumorigenesis. Accordingly, the proteins encoded by the amplified genes are believed to be useful targets for the diagnosis and/or treatment (including prevention) of certain cancers, such as renal cell carcinoma, and may act as predictors of the prognosis of tumor treatment. The present invention is directed to novel methods of diagnosing and treating tumor, such as renal cell carcinoma.

This application is a continuation application claiming priority to U.S. application Ser. No. 10/271,690, filed Oct. 16, 2002, which is a non-provisional application claiming priority to U.S. Provisional Application No. 60/344,534, filed Oct. 18, 2001, which applications are herein incorporated by reference in their entirety.

### FIELD OF THE INVENTION

The present invention relates to methods for the diagnosis and treatment of carcinoma, particularly renal cell carcinoma.

### BACKGROUND OF THE INVENTION

Malignant tumors (cancers) are the second leading cause of death in the United States, after heart disease (Boring et al., CA Cancel J. Clin., 43:7 [1993]).

Cancer is characterized by an increase in the number of abnormal, or neoplastic cells derived from a normal tissue which proliferate to form a tumor mass, the invasion of adjacent tissues by these neoplastic tumor cells, and the generation of malignant cells which eventually spread via the blood or lymphatic system to regional lymph nodes and to distant sites (metastasis). In a cancerous state, a cell proliferates under conditions in which normal cells would not grow. Cancer manifests itself in a wide variety of forms, characterized by different degrees of invasiveness and aggressiveness.

Alteration of gene expression is intimately related to the uncontrolled cell growth and de-differentiation which are a common feature of all cancers. The genomes of certain well studied tumors have been found to show decreased expression of recessive genes, usually referred to as tumor suppression genes, which would normally function to prevent malignant cell growth, and/or overexpression of certain dominant genes, such as oncogenes, that act to promote malignant growth. Each of these genetic changes appears to be responsible for importing some of the traits that, in aggregate, represent the full neoplastic phenotype (Hunter, Cell, 64:1129 [1991] and Bishop, Cell, 64:235-248 [1991]).

A well known mechanism of gene (e.g., oncogene) overexpression in cancer cells is gene amplification. This is a process where in the chromosome of the ancestral cell multiple copies of a particular gene are produced. The process involves unscheduled replication of the region of chromosome comprising the gene, followed by recombination of the replicated segments back into the chromosome (Alitalo et al., Adv. Cancer Res., 47:235-281 [1986]). It is believed that the overexpression of the gene parallels gene amplification, i.e., is proportionate to the number of copies made.

Proto-oncogenes that encode growth factors and growth factor receptors have been identified to play important roles in the pathogenesis of various human malignancies, including breast cancer. For example, it has been found that the human ErbB2 gene (erbB2, also known as her2, or c-erbB-2), which encodes a 185-kd transmembrane glycoprotein receptor (p185^{HER2}) related to the epidermal growth factor receptor EGFR), is overexpressed in about 25% to 30% of human breast cancer (Slamon et al., Science, 235:177-182 [1987]; Slamon et al., Science, 244:707-712 [1989]).

It has been reported that gene amplification of a proto-oncogene is an event typically involved in the more malignant forms of cancer, and could act as a predictor of clinical outcome (Schwab et al., Genes Chromosomes Cancer, 1:181-193 [1990]; Alitalo et al., supra). Thus, erbB2 overexpression is commonly regarded as a predictor of a poor prognosis, especially in patients with primary disease that involves axillary lymph nodes (Slamon et al., [1987] and [1989], supra; Ravdin and Chamness, Gene, 159:19-27 [1995]; and Hynes and Stern, Biochim. Biophys. Acta, 1198:165-184 [1994]), and has been linked to sensitivity and/or resistance to hormone therapy and chemotherapeutic regimens, including CMF (cyclophosphamide, methotrexate, and fluoruracil) and anthracyclines (Baselga et al., Oncology, 11 (3 Suppl1):43-48 [1997]). However, despite the association of erbB2 overexpression with poor prognosis, the odds of HER2-positive patients responding clinically to treatment with taxanes were greater than three times those of HER2-negative patients (Ibid). A recombinant humanized anti-ErbB2 (anti-HER2) monoclonal antibody (a humanized version of the murine anti-ErbB2 antibody 4D5, referred to as rhuMAb HER2 or Herceptin®) has been clinically active in patients with ErbB2-overexpressing metastatic breast cancers that had received extensive prior anticancer therapy. (Baselga et al., J. Clin. Oncol., 14:737-744 [1996]).

Renal cell carcinoma (RCC) is a common solid malignancy and the eleventh leading cause of cancer mortality in the United States. Typically, RCC is a highly vascular neoplasm with an unpredictable pattern of recurrence. Because RCC is a highly vascularized solid malignancy, angiogenesis and tissue invasion may be involved in its pathogenesis. The expression level of several genes have been studied individually and shown to have some correlation with the metastatic potential of RCC. These genes include, for example, fibroblast growth factor (bFGF) (Nanus, D. M. et al., J. Nat'l. Cancer Inst. 85:1597-1599 [1993] and Fujimoto, K. et al., Biochem. Biophys. Res. Commun. 180:386-392 [1991]), vascular endothelial growth factor (VEGF) (Takahashi, A. et al., Cancer Rees. 54:4233-4237 [1994] and Nicol, D. et al., J. Urology 157:1482-1486 [1997]), extracellular matrix-degrading matrix metallopproteinases (MMP-2 and MMP-9) (Kugler, A. et al., J. Urology 160:1914-1918 [1998] and Lein, M. et al., Int'l. J. Cancer 85:801-804 [2000]), angiogenin (Wechsel, H. W. et al., Anticancer Res. 19:1537-1540 [1999]), and cell-to-cell adhesin molecule E-cadherin (Katagiri, A. et al., Br. J. Cancer 71:376-379 [1995]). Additionally, the von Hippel Lindau (VHL) tumor suppressor gene is mutated in sporadic renal cell carcinomas (Knebelmann, G. et al., Cancer Res. 58:226-231 [1998]). The VHL protein is part of an E3 ubiquitin ligase complex and enables proteosomal degradation of the transcription factor, hypoxia inducible factor (HIF-1) (Maxwell, P. H., et al., Nature 399:271-275 [1999]). Mutations in VHL can result in elevated HIF levels and upregulation of hypoxia-induced angiogenic genes such as VEGF. In turn, VEGF mRNA and protein are elevated in tumors compared with normal kidney tissues, and some evidence suggests a relationship to vessel density (Takahashi, A. et al., supra; Nicol, D. et al., supra; and Nakagawa, M. et al., Br. J. Urol. 79:681-687 [1997]). On the other hand, while serum level of VEGF-A protein has been related to cancer grade and stage, evidence for a relationship between VEGF-A and kidney tumor neovascularization is conflicting, suggesting that other angiogenic factors are involved in renal tumor development.

In light of the above, there is obvious interest in identifying novel methods which are useful for diagnosing and treating tumors, such as renal cell carcinomas, which are associated with gene amplification.

### SUMMARY OF THE INVENTION

A. Embodiments

The present invention concerns methods for the diagnosis and treatment of neoplastic cell growth and proliferation in mammals, including humans. The present invention is based on the identification of genes that are amplified in the genome of tumor cells, such as renal cell carcinomas. Such gene amplification is expected to be associated with the overexpression of the gene product and contribute to tumorigenesis. Accordingly, the proteins encoded by the amplified genes are believed to be useful targets for the diagnosis and/or treatment (including prevention) of certain cancers, such as renal cell carcinoma, and may act as predictors of the prognosis of tumor treatment.

In one embodiment, the present invention concerns an isolated antibody which binds to a polypeptide designated herein as CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; and CD36 polypeptide. In one aspect, the isolated antibody specifically binds to a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide. In another aspect, the antibody induces the death of a cell which expresses a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide where such expression is in a tumor cell that overexpresses the polypeptide as compared to a normal cell of the same tissue type. Preferably the tumor cell is in renal cell carcinoma tissue. In yet another aspect, the antibody is a monoclonal antibody, which preferably has non-human complementarity determining region (CDR) residues and human framework region (FR) residues. The antibody may be labeled and may be immobilized on a solid support. In yet another aspect, the antibody is an antibody fragment, a single-chain antibody, or a humanized antibody which binds, preferably specifically, to a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide.

In another embodiment, the invention concerns a composition of matter which comprises an antibody which binds, preferably specifically, to a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide in admixture with a pharmaceutically acceptable carrier. In one aspect, the composition of matter comprises a therapeutically effective amount of the antibody. In another aspect, the composition comprises a further active ingredient, which may, for example, be a further antibody or a cytotoxic or chemotherapeutic agent. Preferably, the composition is sterile.

The invention further concerns antagonists of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide that inhibit one or more of the biological and/or immunological functions or activities of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, such as its angiogenic function in renal cell carcinoma.

In a further embodiment, the invention concerns a method of modulating, preferably inhibiting, transcription and/or translation of the respective amplified gene for treatment of a tumor, such as a renal cell carcinoma. The isolated nucleic acid molecule of the method is RNA or DNA and is the antisense form of the respective gene represented by the nucleic acid sequence provided by the respective GenBank accession number listed in Table 3, where the method involves the application of the antisense nucleic acid to the respective gene and modulation of its transcription and/or translation. Where antagonism of gene expression is desired, the antisense method of the invention preferably down-regulates expression of a gene for which expression is up-regulated in a tumor, such as a RCC. Where up-regulation of a tumor suppressor gene is desired, the antisense method of the invention preferably down-regulates a suppressor of the tumor suppressor gene. Preferably the isolated nucleic acid molecule hybridizes to a nucleic acid molecule encoding a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide or the complement thereof. The isolated nucleic acid molecule is preferably DNA, and hybridization preferably occurs under stringent hybridization and wash conditions. Such nucleic acid molecules can act as antisense molecules of the amplified genes identified herein, which, in turn, can find use in the modulation of the transcription and/or translation of the respective amplified genes, or as antisense primers in amplification reactions. Furthermore, such sequences can be used as part of a method according to the invention in which the gene sequence, or a fragment of at least 20, 50, or 100 nucleic acids of the sequence, are part of a ribozyme and/or a triple helix sequence which, in turn, may be used in regulation of the amplified genes.

In another embodiment, the invention provides a method for determining the presence of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, wherein the method comprises exposing a biological sample, such as a normal or diseased tissue sample (including, but not limited to a tumor sample) to an anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody and determining binding of the antibody to a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide in the sample. In another embodiment, the invention provides a method for determining the presence of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide in a cell, wherein the method comprises exposing the cell to an anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody and determining binding of the antibody to the cell.

In yet another embodiment, the present invention concerns a method of diagnosing tumor in a mammal, such as renal cell carcinoma, comprising detecting the level of expression of a gene encoding a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide (a) in a test sample of tissue cells obtained from the mammal, and (b) in a control sample of known normal tissue cells of the same cell type, wherein a higher expression level in the test sample as compared to the control sample, is indicative of the presence of tumor in the mammal from which the test tissue cells were obtained.

In another embodiment, the present invention concerns a method of diagnosing tumor in a mammal, comprising (a) contacting an anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody with a test sample of tissue cells obtained from the mammal, and (b) detecting the formation of a complex between the anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody and a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide in the test sample, wherein the formation of a complex is indicative of the presence of a tumor in said mammal. The detection may be qualitative or quantitative, and may be performed in comparison with monitoring the complex formation in a control sample of known normal tissue cells of the same cell type. A larger quantity of complexes formed in the test sample indicates the presence of tumor in the mammal from which the test tissue cells were obtained. The antibody preferably carries a detectable label. Complex formation can be monitored, for example, by light microscopy, flow cytometry, fluorimetry, or other techniques known in the art.

The test sample is usually obtained from an individual suspected to have neoplastic cell growth or proliferation (e.g. cancerous cells), such as renal cell carcinoma.

In another embodiment, the present invention concerns a cancer diagnostic kit comprising an anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody and a carrier (e.g., a buffer) in suitable packaging. The kit preferably contains instructions for using the antibody to detect the presence of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide in a sample suspected of containing the same, preferably in a sample of normal or diseased renal tissue, such as a renal cell carcinoma sample.

In yet another embodiment, the invention concerns a method for inhibiting the growth of tumor cells comprising exposing tumor cells which express a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide to an effective amount of an agent which inhibits a biological and/or immunological activity and/or the expression of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, wherein growth of the tumor cells is thereby inhibited. The agent preferably is an anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody, a small organic and inorganic molecule, peptide, phosphopeptide, antisense or ribozyme molecule, or a triple helix molecule. In a specific aspect, the agent, e.g., the anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody, induces cell death. In a further aspect, the tumor cells are further exposed to radiation treatment and/or a cytotoxic or chemotherapeutic agent.

In a further embodiment, the invention concerns an article of manufacture, comprising:

a container;

a label on the container; and

a composition comprising an active agent contained within the container; wherein the composition is effective for inhibiting the growth of tumor cells and the label on the container indicates that the composition can be used for treating conditions characterized by overexpression of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide as compared to a normal cell of the same tissue type, where the condition is preferably renal cell carcinoma. In particular aspects, the active agent in the composition is an agent which inhibits an activity and/or the expression of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide. In preferred aspects, the active agent is an anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody or an antisense oligonucleotide.

The invention also provides a method for identifying a compound that inhibits an activity of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, where the inhibiting activity preferably functions in renal cell carcinoma, the method comprising contacting a candidate compound with a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide under conditions and for a time sufficient to allow these two components to interact and determining whether a biological and/or immunological activity of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide is inhibited. In a specific aspect, either the candidate compound or the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide is immobilized on a solid support. In another aspect, the non-immobilized component carries a detectable label. In a preferred aspect, this method comprises the steps of (a) contacting cells and a candidate compound to be screened in the presence of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide under conditions suitable for the induction of a cellular response normally induced by a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide and (b) determining the induction of said cellular response to determine if the test compound is an effective antagonist.

In another embodiment, the invention provides a method for identifying a compound that inhibits the expression of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide in cells that express the polypeptide, wherein the method comprises contacting the cells with a candidate compound and determining whether the expression of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide is inhibited. In a preferred aspect, this method comprises the steps of (a) contacting cells and a candidate compound to be screened under conditions suitable for allowing expression of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide and (b) determining the inhibition of expression of said polypeptide.

B. Additional Embodiments

In yet another embodiment, the invention concerns antagonists of a native CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide as defined herein. In a particular embodiment, the antagonist is an anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody or a small molecule.

In a further embodiment, the invention concerns a method of identifying antagonists to a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide which comprise contacting the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide with a candidate molecule and monitoring a biological activity mediated by said CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide. Preferably, the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide is a native CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide.

In a still further embodiment, the invention concerns a composition of matter comprising a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide as herein described, or an anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody, in combination with a carrier. Optionally, the carrier is a pharmaceutically acceptable carrier.

Another embodiment of the present invention is directed to the use of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, or an antagonist thereof as hereinbefore described, or an anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody, for the preparation of a medicament useful in the treatment of a condition which is responsive to the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, an antagonist thereof or an anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody. Preferably the condition is renal cell carcinoma.

In other embodiments, the invention provides chimeric molecules comprising any of the herein described polypeptides fused to a heterologous polypeptide or amino acid sequence. Example of such chimeric molecules comprise any of the herein described polypeptides fused to an epitope tag sequence or a Fc region of an immunoglobulin.

In another embodiment, the invention provides an antibody which specifically binds to any of the above or below described polypeptides. Optionally, the antibody is a monoclonal antibody, humanized antibody, antibody fragment or single-chain antibody.

Further embodiments of the present invention will be evident to the skilled artisan upon a reading of the present specification.

### DETAILED DESCRIPTION OF THE INVENTION

I. Definitions

The phrases “gene amplification” and “gene duplication” are used interchangeably and refer to a process by which multiple copies of a gene or gene fragment are formed in a particular cell or cell line. The duplicated region (a stretch of amplified DNA) is often referred to as “amplicon.” Usually, the amount of the messenger RNA (mRNA) produced, i.e., the level of gene expression, also increases in the proportion of the number of copies made of the particular gene expressed.

“Tumor”, as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colorectal cancer, endometrial carcinoma, salivary gland carcinoma, kidney cancer, liver cancer, vulval cancer, thyroid cancer, hepatic carcinoma and various types of head and neck cancer.

“Treatment” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, etc.

“Mammal” for purposes of treatment refers to any animal classified as a mammal, including humans, domestic and farm animals, and zoo, sports, or pet animals, such as dogs, horses, cats, cattle, pigs, sheep, etc. Preferably, the mammal is human.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

Administration “in combination with” one or more further therapeutic agents includes simultaneous (concurrent) and consecutive administration in any order.

The term “cytotoxic agent” as used herein refers to a substance that inhibits or prevents the function of cells and/or causes destruction of cells. The term is intended to include radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin, or fragments thereof.

A “chemotherapeutic agent” is a chemical compound useful in the treatment of cancer. Examples of chemotherapeutic agents include adriamycin, doxorubicin, epirubicin, 5-fluorouracil, cytosine arabinoside (“Ara-C”), cyclophosphamide, thiotepa, busulfan, cytoxin, taxoids, e.g., paclitaxel (Taxol, Bristol-Myers Squibb Oncology, Princeton, N.J.), and doxetaxel (Taxotere, Rhone-Poulenc Rorer, Antony, Rnace), toxotere, methotrexate, cisplatin, melphalan, vinblastine, bleomycin, etoposide, ifosfamide, mitomycin C, mitoxantrone, vincristine, vinorelbine, carboplatin, teniposide, daunomycin, carminomycin, aminopterin, dactinomycin, mitomycins, esperamicins (see U.S. Pat. No. 4,675,187), 5-FU, 6-thioguanine, 6-mercaptopurine, actinomycin D, VP-16, chlorambucil, melphalan, and other related nitrogen mustards. Also included in this definition are hormonal agents that act to regulate or inhibit hormone action on tumors such as tamoxifen and onapristone.

A “growth inhibitory agent” when used herein refers to a compound or composition which inhibits growth of a cell, especially cancer cell overexpressing any of the genes identified herein, either in vitro or in vivo. Thus, the growth inhibitory agent is one which significantly reduces the percentage of cells overexpressing such genes in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxol, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogens, and antineoplastic drugs” by Murakami et al., (WB Saunders: Philadelphia, 1995), especially p. 13.

“Doxorubicin” is an anthracycline antibiotic. The full chemical name of doxorubicin is (8S-cis)-10-[(3-amino-2,3,6-trideoxy-α-L-lyxo-hexapyranosyl)oxy]-7,8,9,10-tetrahydro-6,8,11-trihydroxy-8-(hydroxyacetyl)-1-methoxy-5,12-naphthacenedione.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1a, IL-2, IL-3, IL-4, IL-5, IL-6, IL-7, IL-8, IL-9, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy”, Biochemical Society Transactions, 14:375-382, 615th Meeting, Belfast (1986), and Stella et al., “Prodrugs: A Chemical Approach to Targeted Drug Delivery”, Directed Drug Delivery, Borchardt et al., (ed.), pp. 147-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glysocylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrugs form for use in this invention include, but are not limited to, those chemotherapeutic agents described above.

An “effective amount” of a polypeptide disclosed herein or an antagonist thereof, in reference to inhibition of neoplastic cell growth, tumor growth or cancer cell growth, is an amount capable of inhibiting, to some extent, the growth of target cells. The term includes an amount capable of invoking a growth inhibitory, cytostatic and/or cytotoxic effect and/or apoptosis of the target cells. An “effective amount” of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide antagonist for purposes of inhibiting neoplastic cell growth, tumor growth or cancer cell growth, may be determined empirically and in a routine manner.

A “therapeutically effective amount”, in reference to the treatment of tumor, refers to an amount capable of invoking one or more of the following effects: (1) inhibition, to some extent, of tumor growth, including, slowing down and complete growth arrest; (2) reduction in the number of tumor cells; (3) reduction in tumor size; (4) inhibition (i.e., reduction, slowing down or complete stopping) of tumor cell infiltration into peripheral organs; (5) inhibition (i.e., reduction, slowing down or complete stopping) of metastasis; (6) enhancement of anti-tumor immune response, which may, but does not have to, result in the regression or rejection of the tumor; and/or (7) relief, to some extent, of one or more symptoms associated with the disorder. A “therapeutically effective amount” of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide antagonist for purposes of treatment of tumor may be determined empirically and in a routine manner.

A “growth inhibitory amount” of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37 ; Ephrin A1; Laminin beta 2; Integrin alpha 1; Stanniocalcin 1; Thrombospondin 4; or CD36 polypeptide antagonist is an amount capable of inhibiting the growth of a cell, especially tumor, e.g., cancer cell, either in vitro or in vivo. A “growth inhibitory amount” of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide antagonist for purposes of inhibiting neoplastic cell growth may be determined empirically and in a routine manner.

A “cytotoxic amount” of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide antagonist is an amount capable of causing the destruction of a cell, especially tumor, e.g., cancer cell, either in vitro or in vivo. A “cytotoxic amount” of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide antagonist for purposes of inhibiting neoplastic cell growth may be determined empirically and in a routine manner.

The terms “CXCR4”; “Laminin alpha 4”; “TIMP1”; “Type IV collagen alpha 1”; “Laminin alpha 3”; “Adrenomedullin”; “Thrombospondin 2”; “Type I collagen alpha 2”; “Type VI collagen alpha 2”; “Type VI collagen alpha 3”; “Latent TGFbeta binding protein 2” (“LTBP2”); “Serine or cystein protease inhibitor heat shock protein” (“HSP47”); “Procollagen-lysine, 2-oxoglutarate 5-dioxygenase”; “connexin 43”; “Type IV collagen alpha 2”; “Connexin 37”; “Ephrin A1”; “Laminin beta 2”; “Integrin alpha 1”; “Stanniocalcin 1”; “Thrombospondin 4”; or “CD36” polypeptide or protein when used herein encompass native sequence CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide variants (which are further defined herein). The CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide may be isolated from a variety of sources, such as from human tissue types or from another source, or prepared by recombinant and/or synthetic methods.

A “native sequence CXCR4”; “native sequence Laminin alpha 4”; “native sequence TIMP1”; “native sequence Type IV collagen alpha 1”; “native sequence Laminin alpha 3”; “naive sequence Adrenomedullin”; “native sequence Thrombospondin 2”; “native sequence Type I collagen alpha 2”; “native sequence Type VI collagen alpha 2”; “native sequence Type VI collagen alpha 3”; “native sequence Latent TGFbeta binding protein 2 (“native suquence LTBP2”)”; “native sequence Serine or cystein protease inhibitor heat shock protein (“native sequence HSP47”)”; “native sequence Procollagen-lysine, 2-oxoglutarate 5-dioxygenase”; “native sequence connexin 43”; “native sequence Type IV collagen alpha 2”; “native sequence Connexin 37”; “native sequence Ephrin A1”; “native sequence Laminin beta 2”; “native sequence Integrin alpha 1”; “native sequence Stanniocalcin 1”; “native sequence Thrombospondin 4”; or “native sequence CD36 polypeptide” comprises a polypeptide having the same amino acid sequence as the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide as derived from nature. Such native sequence CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide can be isolated from nature or can be produced by recombinant and/or synthetic means. The term “native sequence” CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 specifically encompasses naturally-occurring truncated or secreted forms (e.g., an extracellular domain sequence), naturally-occurring variant forms (e.g., alternatively spliced forms) and naturally-occurring allelic variants of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptides. In one embodiment of the invention, the native sequence CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide is a mature or full-length native sequence CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide as as encoded by the nucleic acid sequences of the GenBank accession numbers listed in Table 3 for the respective polypeptide. Also, the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptides encoded by the nucleic acid sequences disclosed in the respective GenBank accession numbers listed in Table 3, are shown to begin with the methionine residue designated therein as amino acid position 1, it is conceivable and possible that another methionine residue located either upstream or downstream from amino acid position 1 may be employed as the starting amino acid residue for the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide.

The “extracellular domain” or “ECD” of a polypeptide disclosed herein refers to a form of the polypeptide which is essentially free of the transmembrane and cytoplasmic domains. Ordinarily, a polypeptide ECD will have less than about 1% of such transmembrane and/or cytoplasmic domains and preferably, will have less than about 0.5% of such domains. It will be understood that any transmembrane domain(s) identified for the polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified and as shown in the appended figures. As such, in one embodiment of the present invention, the extracellular domain of a polypeptide of the present invention comprises amino acids 1 to X of the mature amino acid sequence, wherein X is any amino acid within 5 amino acids on either side of the extracellular domain/transmembrane domain boundary.

The approximate location of the “signal peptides” of the various PRO polypeptides disclosed herein are shown in the accompanying figures. It is noted, however, that the C-terminal boundary of a signal peptide may vary, but most likely by no more than about 5 amino acids on either side of the signal peptide C-terminal boundary as initially identified herein, wherein the C-terminal boundary of the signal peptide may be identified pursuant to criteria routinely employed in the art for identifying that type of amino acid sequence element (e.g., Nielsen et al., Prot. Eng., 10:1-6 (1997) and von Heinje et al., Nucl. Acids. Res., 14:4683-4690 (1986)). Moreover, it is also recognized that, in some cases, cleavage of a signal sequence from a secreted polypeptide is not entirely uniform, resulting in more than one secreted species. These mature polypeptides, where the signal peptide is cleaved within no more than about 5 amino acids on either side of the C-terminal boundary of the signal peptide as identified herein, and the polynucleotides encoding them, are contemplated by the present invention.

A “polypeptide variant” of any one of CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide means an active CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide as defined above or below having at least about 80% amino acid sequence identity with a full-length native sequence CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide sequence as disclosed herein, a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide sequence lacking the signal peptide as disclosed herein, an extracellular domain of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, with or without the signal peptide, as disclosed herein or any other fragment of a full-length CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide sequence as disclosed herein. Such CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide variants include, for instance, CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptides wherein one or more amino acid residues are added, or deleted, at the N- or C-terminus of the full-length native amino acid sequence. Ordinarily, a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide variant will have at least about 80% amino acid sequence identity, preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and most preferably at least about 99% amino acid sequence identity with a full-length native sequence CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide sequence as disclosed herein, a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor beat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide sequence lacking the signal peptide as disclosed herein, an extracellular domain of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, with or without the signal peptide, as disclosed herein or any other fragment of a full-length CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide sequence as disclosed herein. Ordinarily, CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 variant polypeptides are at least about 10 amino acids in length, often at least about 20 amino acids in length, more often at least about 30 amino acids in length, more often at least about 40 amino acids in length, more often at least about 50 amino acids in length, more often at least about 60 amino acids in length, more often at least about 70 amino acids in length, more often at least about 80 amino acids in length, more often at least about 90 amino acids in length, more often at least about 100 amino acids in length, more often at least about 150 amino acids in length, more often at least about 200 amino acids in length, more often at least about 300 amino acids in length, or more.

As shown below, Table 1 provides the complete source code for the ALIGN-2 sequence comparison computer program. This source code may be routinely compiled for use on a UNIX operating system to provide the ALIGN-2 sequence comparison computer program.

In addition, following Table 1 are hypothetical exemplifications for using the below described method to determine % amino acid sequence identity and % nucleic acid sequence identity using the ALIGN-2 sequence comparison computer program, wherein “PRO” represents the amino acid sequence of a hypothetical CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cysteinprotease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide of interest, “Comparison Protein” represents the amino acid sequence of a polypeptide against which the “PRO” polypeptide of interest is being compared, “PRO-DNA” represents a hypothetical CXCR4-; Laminin alpha 4-; TIMP1-; Type IV collagen alpha 1-; Laminin alpha 3-; Adrenomedullin-; Thrombospondin 2-; Type I collagen alpha 2-; Type VI collagen alpha 2-; Type VI collagen alpha 3-; Latent TGFbeta binding protein 2- (LTBP2-); Serine or cystein protease inhibitor heat shock protein- (HSP47-); Procollagen-lysine, 2-oxoglutarate 5-dioxygenase-; connexin 43-; Type IV collagen alpha 2-; Connexin 37-; Ephrin A1-; Laminin beta 2-; Integrin alpha 1-; Stanniocalcin 1-; Thrombospondin 4-; or CD36 polypeptide-encoding nucleic acid sequence of interest, “Comparison DNA” represents the nucleotide sequence of a nucleic acid molecule against which the “PRO-DNA” nucleic acid molecule of interest is being compared, “X”, “Y”, and “Z” each represent different hypothetical amino acid residues and “N”, “L” and “V” each represent different hypothetical nucleotides.

Example calculations for determining % amino acid sequence identity and nucleic acid sequence identity:

1.

| | | |
|---|---|---|
| PRO | XXXXXXXXXXXXXXX | (Length = 15 amino acids) |
| Comparison | XXXXXYYYYYYY | (Length = 12 amino acids) |
| Protein | | |

% amino acid sequence identity = (the number of identically matching amino acid residues between the two polypeptide sequences as determined by ALIGN-2) divided by (the total number of amino acid residues of the PRO polypeptide) = 5 divided by 15 = 33.3%

2.

| | | |
|---|---|---|
| PRO | XXXXXXXXXX | (Length = 10 amino acids) |
| Comparison | XXXXXYYYYYYZZYZ | (Length = 15 amino acids) |
| Protein | | |

% amino acid sequence identity = (the number of identically matching amino acid residues between the two polypeptide sequences as determined by ALIGN-2) divided by (the total number of amino acid residues of the PRO polypeptide) = 5 divided by 10 = 50%

3.

| | | |
|---|---|---|
| PRO-DNA | NNNNNNNNNNNNNN | (Length = 14 nucleotides) |
| Comparison | NNNNNNLLLLLLLLLL | (Length = 16 nucleotides) |
| DNA | | |

% nucleic acid sequence identity = (the number of identically matching nucleotides between the two nucleic acid sequences as determined by ALIGN-2) divided by (the total number of nucleotides of the PRO-DNA nucleic acid sequence) = 6 divided by 14 = 42.9%

4.

| | | |
|---|---|---|
| PRO-DNA | NNNNNNNNNNNN | (Length = 12 nucleotides) |
| Comparison | NNNNLLLVV | (Length = 9 nucleotides) |
| DNA | | |

% nucleic acid sequence identity = (the number of identically matching nucleotides between the two nucleic acid sequences as determined by ALIGN-2) divided by (the total number of nucleotides of the PRO-DNA nucleic acid sequence) = 4 divided by 12 = 33.3%

“Percent (%) amino acid sequence identity” with respect to the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide sequences identified herein is defined as the percentage of amino acid residues in a candidate sequence that are identical with the amino acid residues in a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained as described below by using the sequence comparison computer program ALIGN-2, wherein the complete source code for the ALIGN-2 program is provided in Table 1. The ALIGN-2 sequence comparison computer program was authored by Genentech, Inc., and the source code shown in Table 1 has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1. The ALIGN-2 program should be compiled for use on a UNIX operating system, preferably digital UNIX V4.0D. All sequence comparison parameters are set by the ALIGN-2 program and do not vary.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations, Tables 2A-2B demonstrate how to calculate the % amino acid sequence identity of the amino acid sequence designated “Comparison Protein” to the amino acid sequence designated “PRO”.

Unless specifically stated otherwise, all % amino acid sequence identity values used herein are obtained as described above using the ALIGN-2 sequence comparison computer program. However, % amino acid sequence identity may also be determined using the sequence comparison program NCBI-BLAST2 (Altschul et al., Nucleic Acids Res., 25:3389-3402 (1997)). The NCBI-BLAST2 sequence comparison program may be downloaded from http://www.ncbi.nlm.nih.gov. NCBI-BLAST2 uses several search parameters, wherein all of those search parameters are set to default values including, for example, unmask=yes, strand=all, expected occurrences=10, minimum low complexity length= 15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

In addition, % amino acid sequence identity may also be determined using the WU-BLAST-2 computer program (Altschul et al., Methods in Enzymology, 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. For purposes herein, a % amino acid sequence identity value is determined by dividing (a) the number of matching identical amino acids residues between the amino acid sequence of the PRO polypeptide of interest having a sequence derived from the native PRO polypeptide and the comparison amino acid sequence of interest (i.e., the sequence against which the PRO polypeptide of interest is being compared which may be a PRO variant polypeptide) as determined by WU-BLAST-2 by (b) the total number of amino acid residues of the PRO polypeptide of interest. For example, in the statement “a polypeptide comprising an amino acid sequence A which has or having at least 80% amino acid sequence identity to the amino acid sequence B”, the amino acid sequence A is the comparison amino acid sequence of interest and the amino acid sequence B is the amino acid sequence of the PRO polypeptide of interest.

“CXCR4 variant polynucleotide”; “Laminin alpha 4 variant polynucleotide”; “TIMP1; Type IV collagen alpha 1 variant polynucleotide”; “Laminin alpha 3 variant polynucleotide”; “Adrenomedullin variant polynucleotide”; “Thrombospondin 2 variant polynucleotide”; “Type I collagen alpha 2 variant polynucleotide”; “Type VI collagen alpha 2 variant polynucleotide”; “Type VI collagen alpha 3 variant polynucleotide”; “Latent TGFbeta binding protein 2 variant polynucleotide” (“LTBP2 variant polynucleotide”); “Serine or cystein protease inhibitor heat shock protein variant polynucleotide” (“HSP47 variant polynucleotide”); “Procollagen-lysine, 2-oxoglutarate 5-dioxygenase variantpolynucleotide”; “connexin 43 variant polynucleotide”; “Type IV collagen alpha 2 variant polynucleotide”; “Connexin 37 variant polynucleotide”; “Ephrin A1 variant polynucleotide”; “Laminin beta 2 variant polynucleotide”; “Integrin alpha 1 variant polynucleotide”; “Stanniocalcin 1 variant polynucleotide”; “Thrombospondin 4 variant polynucleotide”; or “CD36 variant polynucleotide” or “CXCR4 variant polynucleotide”; “Laminin alpha 4 variant polynucleotide”; “TIMP1; Type IV collagen alpha 1 variant polynucleotide”; “Laminin alpha 3 variant polynucleotide”; “Adrenomedullin variant polynucleotide”; “Thrombospondin 2 variant polynucleotide”; “Type I collagen alpha 2 variant polynucleotide”; “Type VI collagen alpha 2 variant polynucleotide”; “Type VI collagen alpha 3 variant polynucleotide”; “Latent TGFbeta binding protein 2 variant polynucleotide” (“LTBP2 variant polynucleotide”); “Serine or cystein protease inhibitor heat shock protein variant polynucleotide” (“HSP47 variant polynucleotide”); “Procollagen-lysine, 2-oxoglutarate 5-dioxygenase variant polynucleotide”; “connexin 43 variant polynucleotide”; “Type IV collagen alpha 2 variant polynucleotide”; “Connexin 37 variant polynucleotide”; “Ephrin A1 variant polynucleotide”; “Laminin beta 2 variant polynucleotide”; “Integrin alpha 1 variant polynucleotide”; “Stanniocalcin 1 variant polynucleotide”; “Thrombospondin 4 variant polynucleotide”; or “CD36 variant polynucleotide” means a nucleic acid molecule which encodes an active CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide as defined below and which has at least about 80% nucleic acid sequence identity with a nucleotide acid sequence encoding a full-length native sequence CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide sequence as disclosed herein, a full-length native sequence CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide sequence lacking the signal peptide as disclosed herein, an extracellular domain of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, with or without the signal peptide, as disclosed herein or any other fragment of a full-length CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide sequence as disclosed herein. Ordinarily, a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 variant polynucleotide will have at least about 80% nucleic acid sequence identity, more preferably at least about 81% nucleic acid sequence identity, more preferably at least about 82% nucleic acid sequence identity, more preferably at least about 83% nucleic acid sequence identity, more preferably at least about 84% nucleic acid sequence identity, more preferably at least about 85% nucleic acid sequence identity, more preferably at least about 86% nucleic acid sequence identity, more preferably at least about 87% nucleic acid sequence identity, more preferably at least about 88% nucleic acid sequence identity, more preferably at least about 89% nucleic acid sequence identity, more preferably at least about 90% nucleic acid sequence identity, more preferably at least about 91% nucleic acid sequence identity, more preferably at least about 92% nucleic acid sequence identity, more preferably at least about 93% nucleic acid sequence identity, more preferably at least about 94% nucleic acid sequence identity, more preferably at least about 95% nucleic acid sequence identity, more preferably at least about 96% nucleic acid sequence identity, more preferably at least about 97% nucleic acid sequence identity, more preferably at least about 98% nucleic acid sequence identity and yet more preferably at least about 99% nucleic acid sequence identity with the nucleic acid sequence encoding a full-length native sequence CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide sequence as disclosed herein, a full-length native sequence CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide sequence lacking the signal peptide as disclosed herein, an extracellular domain of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, with or without the signal sequence, as disclosed herein or any other fragment of a full-length CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide sequence as disclosed herein. Variants do not encompass the native nucleotide sequence.

Ordinarily, CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; and CD36 variant polynucleotides are at least about 30 nucleotides in length, often at least about 60 nucleotides in length, more often at least about 90 nucleotides in length, more often at least about 120 nucleotides in length, more often at least about 150 nucleotides in length, more often at least about 180 nucleotides in length, more often at least about 210 nucleotides in length, more often at least about 240 nucleotides in length, more often at least about 270 nucleotides in length, more often at least about 300 nucleotides in length, more often at least about 450 nucleotides in length, more often at least about 600 nucleotides in length, more often at least about 900 nucleotides in length, or more.

“Percent (%) nucleic acid sequence identity” with respect to the CXCR4; Laminin alpha 4; TIMP1; Type IV collagen alp “CXCR4 variant polypeptide”; “Laminin alpha 4 variant polypeptide”; “TIMP1; Type IV collagen alpha 1 variant polypeptide”; “Laminin alpha 3 variant polypeptide”; “Adrenomedullin variant polypeptide”; “Thrombospondin 2 variant polypeptide”; “Type I collagen alpha 2 variant polypeptide”; “Type VI collagen alpha 2 variant polypeptide”; “Type VI collagen alpha 3 variant polypeptide”; “Latent TGFbeta binding protein 2 variant polypeptide” (“LTBP2 variant polypeptide”); “Serine or cystein protease inhibitor heat shock protein variant polypeptide” (“HSP47 variant polypeptide”); “Procollagen-lysine, 2-oxoglutarate 5-dioxygenase variant polypeptide”; “connexin 43 variant polypeptide”; “Type IV collagen alpha 2 variant polypeptide”; “Connexin 37 variant polypeptide”; “Ephrin A1 variant polypeptide”; “Laminin beta 2 variant polypeptide”; “Integrin alpha 1 variant polypeptide”; “Stanniocalcin 1 variant polypeptide”; “Thrombospondin 4 variant polypeptide”; or “CD36 variant polypeptide” ha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide-encoding nucleic acid sequences identified herein is defined as the percentage of nucleotides in a candidate sequence that are identical with the nucleotides in a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide-encoding nucleic acid sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % nucleic acid sequence identity values are obtained as described below by using the sequence comparison computer program ALIGN-2, wherein the complete source code for the ALIGN-2 program is provided in Table 1. The ALIGN-2 sequence comparison computer program was authored by Genentech, Inc., and the source code shown in Table 1 has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1. The ALIGN-2 program should be compiled for use on a UNIX operating system, preferably digital UNIX V4.0D. All sequence comparison parameters are set by the ALIGN-2 program and do not vary.

For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:
100 times the fraction W/Z
where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. As examples of % nucleic acid sequence identity calculations, Tables 2C-2D demonstrate how to calculate the % nucleic acid sequence identity of the nucleic acid sequence designated “Comparison DNA” to the nucleic acid sequence designated “PRO-DNA”.

Unless specifically stated otherwise, all % nucleic acid sequence identity values used herein are obtained as described above using the ALIGN-2 sequence comparison computer program. However, % nucleic acid sequence identity may also be determined using the sequence comparison program NCBI-BLAST2 (Altschul et al., Nucleic Acids Res., 25:3389-3402 (1997)). The NCBI-BLAST2 sequence comparison program may be downloaded from http://www.ncbi.nlm.nih.gov. NCBI-BLAST2 uses several search parameters, wherein all of those search parameters are set to default values including, for example, unmask=yes, strand=all, expected occurrences=10, minimum low complexity length= 15/5, multi-pass e-value=0.01, constant for multi-pass=25, dropoff for final gapped alignment=25 and scoring matrix=BLOSUM62.

In situations where NCBI-BLAST2 is employed for sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:
100 times the fraction W/Z
where W is the number of nucleotides scored as identical matches by the sequence alignment program NCBI-BLAST2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

In addition, % nucleic acid sequence identity values may also be generated using the WU-BLAST-2 computer program (Altschul et al., Methods in Enzymology, 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. For purposes herein, a % nucleic acid sequence identity value is determined by dividing (a) the number of matching identical nucleotides between the nucleic acid sequence of the PRO polypeptide-encoding nucleic acid molecule of interest having a sequence derived from the native sequence PRO polypeptide-encoding nucleic acid and the comparison nucleic acid molecule of interest (i.e., the sequence against which the PRO polypeptide-encoding nucleic acid molecule of interest is being compared which may be a variant PRO polynucleotide) as determined by WU-BLAST-2 by (b) the total number of nucleotides of the PRO polypeptide-encoding nucleic acid molecule of interest. For example, in the statement “an isolated nucleic acid molecule comprising a nucleic acid sequence A which has or having at least 80% nucleic acid sequence identity to the nucleic acid sequence B”, the nucleic acid sequence A is the comparison nucleic acid molecule of interest and the nucleic acid sequence B is the nucleic acid sequence of the PRO polypeptide-encoding nucleic acid molecule of interest.

In other embodiments, CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 variant polynucleotides are nucleic acid molecules that encode an active CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide and which are capable of hybridizing, preferably under stringent hybridization and wash conditions, to nucleotide sequences encoding the full-length CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, which nucleotide sequences are found in the GenBank accession numbers listed in Table 3 for the respective polypeptides. CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cysteinprotease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 variant polypeptides may be those that are encoded by a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 variant polynucleotide.

The term “positives”, in the context of the amino acid sequence identity comparisons performed as described above, includes amino acid residues in the sequences compared that are not only identical, but also those that have similar properties. Amino acid residues that score a positive value to an amino acid residue of interest are those that are either identical to the amino acid residue of interest or are a preferred substitution (as defined in Table 3 below) of the amino acid residue of interest.

For purposes herein, the % value of positives of a given amino acid sequence A to, with, or against a given amino acid sequence B (which can alternatively be phrased as a given amino acid sequence A that has or comprises a certain % positives to, with, or against a given amino acid sequence B) is calculated as follows:
100 times the fraction X/Y
where X is the number of amino acid residues scoring a positive value as defined above by the sequence alignment program ALIGN-2 in that program's alignment of A and B, and where Y is the total number of amino acid residues in B. It will be appreciated that where the length of amino acid sequence A is not equal to the length of amino acid sequence B, the % positives of A to B will not equal the % positives of B to A.

“Isolated,” when used to describe the various polypeptides disclosed herein, means polypeptide that has been identified and separated and/or recovered from a component of its natural environment. Preferably, the isolated polypeptide is free of association with all components with which it is naturally associated. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 natural environment will not be present. Ordinarily, however, isolated polypeptide will be prepared by at least one purification step.

An “isolated” nucleic acid molecule encoding a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide or an “isolated” nucleic acid encoding an anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody, is a nucleic acid molecule that is identified and separated from at least one contaminant nucleic acid molecule with which it is ordinarily associated in the natural source of the CXCR4-; Laminin alpha 4-; TIMP1-; Type IV collagen alpha 1-; Laminin alpha 3-; Adrenomedullin-; Thrombospondin 2-; Type I collagen alpha 2-; Type VI collagen alpha 2-; Type VI collagen alpha 3-; Latent TGFbeta binding protein 2- (LTBP2-); Serine or cystein protease inhibitor heat shock protein- (HSP47-); Procollagen-lysine, 2-oxoglutarate 5-dioxygenase-; connexin 43-; Type IV collagen alpha 2-; Connexin 37-; Ephrin A1-; Laminin beta 2-; Integrin alpha 1-; Stanniocalcin 1-; Thrombospondin 4-; or CD36-encoding nucleic acid or the anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide -encoding nucleic acid. Preferably, the isolated nucleic acid is free of association with all components with which it is naturally associated. An isolated CXCR4-; Laminin alpha 4-; TIMP1-; Type IV collagen alpha 1-; Laminin alpha 3-; Adrenomedullin-; Thrombospondin 2-; Type I collagen alpha 2-; Type VI collagen alpha 2-; Type VI collagen alpha 3-; Latent TGFbeta binding protein 2- (LTBP2-); Serine or cystein protease inhibitor heat shock protein- (HSP47-); Procollagen-lysine, 2-oxoglutarate 5-dioxygenase-; connexin 43-; Type IV collagen alpha 2-; Connexin 37-; Ephrin A1-; Laminin beta 2-; Integrin alpha 1-; Stanniocalcin 1-; Thrombospondin 4-; or CD36 polypeptide-encoding nucleic acid molecule or an anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide-encoding nucleic acid molecule is other than in the form or setting in which it is found in nature. Isolated nucleic acid molecules therefore are distinguished from the CXCR4-; Laminin alpha 4-; TIMP1-; Type IV collagen alpha 1-; Laminin alpha 3-; Adrenomedullin-; Thrombospondin 2-; Type I collagen alpha 2-; Type VI collagen alpha 2-; Type VI collagen alpha 3-; Latent TGFbeta binding protein 2- (LTBP2-); Serine or cystein protease inhibitor heat shock protein- (HSP47-); Procollagen-lysine, 2-oxoglutarate 5-dioxygenase-; connexin 43-; Type IV collagen alpha 2-; Connexin 37-; Ephrin A1-; Laminin beta 2-; Integrin alpha 1-; Stanniocalcin 1-; Thrombospondin 4-; or CD36-encoding nucleic acid molecule or the anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cysteinprotease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide-encoding nucleic acid molecule as it exists in natural cells. However, an isolated nucleic acid molecule encoding a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide or an anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody includes CXCR4-; Laminin alpha 4-; TIMP1-; Type IV collagen alpha 1-; Laminin alpha 3-; Adrenomedullin-; Thrombospondin 2-; Type I collagen alpha 2-; Type VI collagen alpha 2-; Type VI collagen alpha 3-; Latent TGFbeta binding protein 2- (LTBP2-); Serine or cystein protease inhibitor heat shock protein- (HSP47-); Procollagen-lysine, 2-oxoglutarate 5-dioxygenase-; connexin 43-; Type IV collagen alpha 2-; Connexin 37-; Ephrin A1-; Laminin beta 2-; Integrin alpha 1-; Stanniocalcin 1-; Thrombospondin 4-; or CD36-nucleic acid molecules and anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide -encoding nucleic acid molecules contained in cells that ordinarily express CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptides or express anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibodies where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “antibody” is used in the broadest sense and specifically covers, for example, single anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide monoclonal antibodies (including antagonist, and neutralizing antibodies), anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody compositions with polyepitopic specificity, single chain anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibodies, and fragments of anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibodies (see below). The term “monoclonal antibody” as used herein refers to an antibody obtained from a population of substantially homogeneous antibodies, i.e., the individual antibodies comprising the population are identical except for possible naturally-occurring mutations that may be present in minor amounts.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0. 1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent than those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 35° C.-50° C. The skilled artisan will recognize how to adjust the temperature, ionic strength, etc. as necessary to accommodate factors such as probe length and the like.

The term “epitope tagged” when used herein refers to a chimeric polypeptide comprising a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

“Active” or “activity” for the purposes herein refers to form(s) of CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptides which retain a biological and/or an immunological activity/property of a native or naturally-occurring CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, wherein “biological” activity refers to a function (either inhibitory or stimulatory) caused by a native or naturally-occurring CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide other than the ability to induce the production of an antibody against an antigenic epitope possessed by a a native or naturally-occurring CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide and an “immunological” activity refers to the ability to induce the production of an antibody against an antigenic epitope possessed by a native or naturally-occurring CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide.

“Biological activity” in the context of an antibody or another antagonist molecule that can be identified by the screening assays disclosed herein (e.g., an organic or inorganic small molecule, peptide, etc.) is used to refer to the ability of such molecules to bind or complex with the polypeptides encoded by the amplified genes identified herein, or otherwise interfere with the interaction of the encoded polypeptides with other cellular proteins or otherwise interfere with the transcription or translation of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide. “Biological activity” in the context of an agonist molecule that enhances the activity of, for example, native anti-angiogenic molecules refers to the ability of such molecules to bind or complex with the polypeptides encoded by the amplified genes identified herein or otherwise modify the interaction of the encoded polypeptides with other cellular proteins or otherwise enhance the transcription or translation of a TIMP1 or thrombospondin 2 polypeptide. A preferred biological activity is growth inhibition of a target tumor cell. Another preferred biological activity is cytotoxic activity resulting in the death of the target tumor cell.

The term “biological activity” in the context of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide means the ability of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide to induce neoplastic cell growth or uncontrolled cell growth.

The phrase “immunological activity” means immunological cross-reactivity with at least one epitope of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide.

“Immunological cross-reactivity” as used herein means that the candidate polypeptide is capable of competitively inhibiting the qualitative biological activity of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide having this activity with polyclonal antisera raised against the known active CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide. Such antisera are prepared in conventional fashion by injecting goats or rabbits, for example, subcutaneously with the known active analogue in complete Freund's adjuvant, followed by booster intraperitoneal or subcutaneous injection in incomplete Freunds. The immunological cross-reactivity preferably is “specific”, which means that the binding affinity of the immunologically cross-reactive molecule (e.g., antibody) identified, to the corresponding CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide is significantly higher (preferably at least about 2-times, more preferably at least about 4-times, even more preferably at least about 8-times, most preferably at least about 10-times higher) than the binding affinity of that molecule to any other known native polypeptide.

The term “antagonist” is used in the broadest sense, and includes any molecule that partially or fully blocks, inhibits, or neutralizes a biological activity of a native CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide disclosed herein or the transcription or translation thereof. Suitable antagonist molecules specifically include antagonist antibodies or antibody fragments, fragments, peptides, small organic molecules, anti-sense nucleic acids, etc. Included are methods for identifying antagonists of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide with a candidate antagonist molecule and measuring a detectable change in one or more biological activities normally associated with the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide.

A “small molecule” is defined herein to have a molecular weight below about 500 Daltons.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas. The term “antibody” is used in the broadest sense and specifically covers, without limitation, intact monoclonal antibodies, polyclonal antibodies, multispecific antibodies (e.g., bispecific antibodies) formed from at least two intact antibodies, and antibody fragments so long as they exhibit the desired biological activity.

“Native antibodies” and “native immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR) regions. The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al., NIH Publ. No.91-3242, Vol. I, pages 647-669 (1991)). The constant domains are not involved directly in binding an antibody to an antigen, but exhibit various effector functions, such as participation of the antibody in antibody-dependent cellular toxicity.

The term “hypervariable region” when used herein refers to the amino acid residues of an antibody which are responsible for antigen-binding. The hypervariable region comprises amino acid residues from a “complementarity determining region” or “CDR” (i.e., residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institute of Health, Bethesda, Md. [1991]) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Clothia and Lesk, J. Mol. Biol., 196:901-917 [1987]). “Framework” or “FR” residues are those variable domain residues other than the hypervariable region residues as herein defined.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng., 8(10):1057-1062 [1995]); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called α, δ, ε, γ, and μ, respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 [1975], or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 [1991] and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 [1984]).

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a CDR of the recipient are replaced by residues from a CDR of a non-human species (donor antibody) such as mouse, rat or rabbit having the desired specificity, affinity, and capacity. In some instances, Fv FR residues of the human immunoglobulin are replaced by corresponding non-human residues. Furthermore, humanized antibodies may comprise residues which are found neither in the recipient antibody nor in the imported CDR or framework sequences. These modifications are made to further refine and maximize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see, Jones et al., Nature, 321:522-525 (1986); Reichmann et al., Nature, 332:323-329 [1988]; and Presta, Curr. Op. Struct. Biol., 2:593-596 (1992). The humanized antibody includes a PRIMATIZED™ antibody wherein the antigen-binding region of the antibody is derived from an antibody produced by immunizing macaque monkeys with the antigen of interest.

“Single-chain Fv” or “sFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The word “label” when used herein refers to a detectable compound or composition which is conjugated directly or indirectly to the antibody so as to generate a “labeled” antibody. The label may be detectable by itself (e.g., radioisotope labels or fluorescent labels) or, in the case of an enzymatic label, may catalyze chemical alteration of a substrate compound or composition which is detectable. Radionuclides that can serve as detectable labels include, for example, I-131, I-123, I-125, Y-90, Re-188, Re-186, At-211, Cu-67, Bi-212, and Pd-109. The label may also be a non-detectable entity such as a toxin.

By “solid phase” is meant a non-aqueous matrix to which the antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide or antibody thereto and, optionally, a chemotherapeutic agent) to a mammal. The components of the liposome are commonly arranged in a bilayer formation, similar to the lipid arrangement of biological membranes.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

I. Methods of the Invention To carry out the methods of the invention, it may be useful to prepare native polypeptide sequences or variants of the genes of interest as well as antibodies. The native polypeptide sequences are disclosed in the GenBank accession numbers listed in Table 3. Non-limiting procedures useful for carrying out the invention are provided below.

A. Amino acid sequence variants of the polypetides of interest: Where variants are contemplated of the polypeptides of interest or antibodies that bind to them, conservative amino acid substitutions of interest are shown in Table 2 under the heading of preferred substitutions. If such substitutions result in a change in biological activity, then more substantial changes, denominated exemplary substitutions in Table 2, or as further described below in reference to amino acid classes, are introduced and the products screened.

TABLE 2

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

Substantial modifications in function or immunological identity of the polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244: 1081-1085 (1989)]. Alanine is also typically preferred because it is the most common amino acid. Further, it is frequently found in both buried and exposed positions [Creighton, The Proteins, (W. H. Freeman & Co., N.Y.); Chothia, J. Mol. Biol., 150:1 (1976)]. If alanine substitution does not yield adequate amounts of variant, an isoteric amino acid can be used.

Another type of modification of CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide comprises linking the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

The CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 of the present invention may also be modified in a way to form a chimeric molecule comprising CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 fused to another, heterologous polypeptide or amino acid sequence.

In one embodiment, such a chimeric molecule comprises a fusion of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 with a tag polypeptide which provides an epitope to which an anti-tag antibody can selectively bind. The epitope tag is generally placed at the amino- or carboxyl-terminus of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36. The presence of such epitope-tagged forms of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 can be detected using an antibody against the tag polypeptide. Also, provision of the epitope tag enables the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 to be readily purified by affinity purification using an anti-tag antibody or another type of affinity matrix that binds to the epitope tag. Various tag polypeptides and their respective antibodies are well known in the art. Examples include poly-histidine (poly-His) or poly-histidine-glycine (poly-His-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In an alternative embodiment, the chimeric molecule may comprise a fusion of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also, U.S. Pat. No. 5,428,130 issued Jun. 27, 1995.

B. Preparation of CXCR4; Laminin Alpha 4; TIMP1; Type IV Collagen Alpha 1; Laminin Alpha 3; Adrenomedullin; Thrombospondin 2; Type I Collagen Alpha 2; Type VI Collagen Alpha 2; Type VI Collagen Alpha 3; Latent TGFbeta Binding Protein 2 (LTBP2); Serine or Cystein Protease Inhibitor Heat Shock Protein (HSP47); Procollagen-Lysine, 2-Oxoglutarate 5-Dioxygenase; Connexin 43; Type IV Collagen Alpha 2; Connexin 37; Ephrin A1; Laminin Beta 2; Integrin Alpha 1; Stanniocalcin 1; Thrombospondin 4; or CD36 Polypeptides

The description below relates primarily to production of CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 by culturing cells transformed or transfected with a vector containing CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 nucleic acid. It is, of course, contemplated that alternative methods, which are well known in the art, may be employed to prepare CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36. For instance, the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 sequence, or portions thereof, may be produced by direct peptide synthesis using solid-phase techniques [see, e.g., Stewart et al., Solid-Phase Peptide Synthesis, W. H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 may be chemically synthesized separately and combined using chemical or enzymatic methods to produce the full-length CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36.

C. Isolation of DNA Encoding a CXCR4; Laminin Alpha 4; TIMP1; Type IV Collagen Alpha 1; Laminin Alpha 3; Adrenomedullin; Thrombospondin 2; Type I Collagen Alpha 2; Type VI Collagen Alpha 2; Type VI Collagen Alpha 3; Latent TGFbeta Binding Protein 2 (LTBP2); Serine or Cystein Protease Inhibitor Heat Shock Protein (HSP47); Procollagen-Lysine, 2-Oxoglutarate 5-Dioxygenase; Connexin 43; Type IV Collagen Alpha 2; Connexin 37; Ephrin A1; Laminin Beta 2; Integrin Alpha 1; Stanniocalcin 1; Thrombospondin 4; or CD36 Polypeptide

DNA encoding CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 may be obtained from a cDNA library prepared from tissue believed to possess the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 mRNA and to express it at a detectable level. Accordingly, human CXCR4; human Laminin alpha 4; human TIMP1; human Type IV collagen alpha 1; human Laminin alpha 3; human Adrenomedullin; human Thrombospondin 2; human Type I collagen alpha 2; human Type VI collagen alpha 2; human Type VI collagen alpha 3; human Latent TGFbeta binding protein 2 (human LTBP2); human Serine or cystein protease inhibitor heat shock protein (human HSP47); human Procollagen-lysine, 2-oxoglutarate 5-dioxygenase; human connexin 43; human Type IV collagen alpha 2; human Connexin 37; human Ephrin A1; human Laminin beta 2; human Integrin alpha 1; human Stanniocalcin 1; human Thrombospondin 4; or human CD36 DNA can be conveniently obtained from a cDNA library prepared from human tissue, such as described in the Examples. CXCR4-; Laminin alpha 4-; TIMP1-; Type IV collagen alpha 1-; Laminin alpha 3-; Adrenomedullin-; Thrombospondin 2-; Type I collagen alpha 2-; Type VI collagen alpha 2-; Type VI collagen alpha 3-; Latent TGFbeta binding protein 2 (LTBP2)-; Serine or cystein protease inhibitor heat shock protein (HSP47)-; Procollagen-lysine, 2-oxoglutarate 5-dioxygenase-; connexin 43-; Type IV collagen alpha 2-; Connexin 37-; Ephrin A1-; Laminin beta 2-; Integrin alpha 1-; Stanniocalcin 1-; Thrombospondin 4-; or CD36-encoding gene may also be obtained from a genomic library or by oligonucleotide synthesis.

Libraries can be screened with probes (such as antibodies to the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding PRO381, PRO1269, PRO1410, PRO1755, PRO1780, PRO1788, PRO3434, PRO1927, PRO3567, PRO1295, PRO1293, PRO1303, PRO4344, PRO4354, PRO4397, PRO4407, PRO1555, PRO1096, PRO2038 or PRO2262 is to use PCR methodology [Sambrook et al., supra; Dieffenbach et al., PCR Primer: A Laboratory Manual (Cold Spring Harbor Laboratory Press, 1995)].

The Examples below describe techniques for screening a cDNA library. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

D. Selection and Transformation of Host Cells

Host cells are transfected or transformed with expression or cloning vectors described herein for CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see, Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and E. coli strain K5772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan^{r}; E. coli W3110 strain 37D6, which has the complete genotype tonA ptr3 phoA E15 (argF-lac) 169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for CXCR4-; Laminin alpha 4-; TIMP1-; Type IV collagen alpha 1-; Laminin alpha 3-; Adrenomedullin-; Thrombospondin 2-; Type I collagen alpha 2-; Type VI collagen alpha 2-; Type VI collagen alpha 3-; Latent TGFbeta binding protein 2 (LTBP2)-; Serine or cystein protease inhibitor heat shock protein (HSP47)-; Procollagen-lysine, 2-oxoglutarate 5-dioxygenase-; connexin 43-; Type IV collagen alpha 2-; Connexin 37-; Ephrin A1-; Laminin beta 2-; Integrin alpha 1-; Stanniocalcin 1-; Thrombospondin 4-; or CD36-encoding vectors. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9: 968-975 (1991)) such as, e.g., K. lactis (MW98-8C, CBS683, CBS4574; Louvencourt et al., J. Bacteriol., 737 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Vanden Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81:1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/-DHFR (CHO), Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

E. Selection and Use of a Replicable Vector

The nucleic acid (e.g., cDNA or genomic DNA) encoding CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type V collagen alpha 2; Connexin 37; Ephrin A1; Laminin beta 2; Integrin alpha 1; Stanniocalcin 1; Thrombospondin 4; or CD36 may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the CXCR4-; Laminin alpha 4-; TIMP1-; Type IV collagen alpha 1-; Laminin alpha 3-; Adrenomedullin-; Thrombospondin 2-; Type I collagen alpha 2-; Type VI collagen alpha 2-; Type VI collagen alpha 3-; Latent TGFbeta binding protein 2 (LTBP2)-; Serine or cystein protease inhibitor heat shock protein (HSP47)-; Procollagen-lysine, 2-oxoglutarate 5-dioxygenase-; connexin 43-; Type IV collagen alpha 2-; Connexin 37-; Ephrin A1-; Laminin beta 2-; Integrin alpha 1-; Stanniocalcin 1-; Thrombospondin 4-; or CD36-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the CXCR4-; Laminin alpha 4-; TIMP1-; Type IV collagen alpha 1-; Laminin alpha 3-; Adrenomedullin-; Thrombospondin 2-; Type I collagen alpha 2-; Type VI collagen alpha 2-; Type VI collagen alpha 3-; Latent TGFbeta binding protein 2 (LTBP2)-; Serine or cystein protease inhibitor heat shock protein (HSP47)-; Procollagen-lysine, 2-oxoglutarate 5-dioxygenase-; connexin 43-; Type IV collagen alpha 2-; Connexin 37-; Ephrin A1-; Laminin beta 2-; Integrin alpha 1-; Stanniocalcin 1-; Thrombospondin 4-; or CD36-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene, 7:141 (1979); Tschemper et al., Gene, 10:157 (1980)]. The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the CXCR4-; Laminin alpha 4-; TIMP1-; Type IV collagen alpha 1-; Laminin alpha 3-; Adrenomedullin-; Thrombospondin 2-; Type I collagen alpha 2-; Type VI collagen alpha 2-; Type VI collagen alpha 3-; Latent TGFbeta binding protein 2 (LTBP2)-; Serine or cystein protease inhibitor heat shock protein (HSP47)-; Procollagen-lysine, 2-oxoglutarate 5-dioxygenase-; connexin 43-; Type IV collagen alpha 2-; Connexin 37-; Ephrin A1-; Laminin beta 2-; Integrin alpha 1-; Stanniocalcin 1-; Thrombospondin 4-; or CD36-encoding nucleic acid sequence to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgarno (S.D.) sequence operably linked to the DNA encoding CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36.

Still other methods, vectors, and host cells suitable for adaptation to the synthesis of CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1 ; Thrombospondin 4; or CD36 in recombinant vertebrate cell culture are described in Gething et al., Nature, 293:620-625 (1981); Mantei et al., Nature, 281:40-46 (1979); EP 117,060; and EP 117,058.

F. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide or against a synthetic peptide based on the DNA sequences provided herein or against an exogenous sequence fused to CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 DNA and encoding a specific antibody epitope.

G. Purification of Polypeptide

Forms of CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 may be recovered from culture medium or from host cell lysates. If membrane-bound, it can be released from the membrane using a suitable detergent solution (e.g., Triton-X 100) or by enzymatic cleavage. Cells employed in expression of CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 can be disrupted by various physical or chemical means, such as freeze-thaw cycling, sonication, mechanical disruption, or cell lysing agents.

It may be desired to purify CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 produced.

H. Amplification of Genes Encoding the CXCR4; Laminin Alpha 4; TIMP1; Type IV Collagen Alpha 1; Laminin Alpha 3; Adrenomedullin; Thrombospondin 2; Type I Collagen Alpha 2; Type VI Collagen Alpha 2; Type VI Collagen Alpha 3; Latent TGFbeta Binding Protein 2 (LTBP2); Serine or Cystein Protease Inhibitor Heat Shock Protein (HSP47); Procollagen-Lysine, 2-Oxoglutarate 5-Dioxygenase; Connexin 43; Type IV Collagen Alpha 2; Connexin 37; Ephrin A1; Laminin Beta 2; Integrin Alpha 1; Stanniocalcin 1; Thrombospondin 4; or CD36 Polypeptides in Tumor Tissues and Cell Lines

The present invention is based on the identification and characterization of genes that are amplified in certain cancer cells.

The genome of prokaryotic and eukaryotic organisms is subjected to two seemingly conflicting requirements. One is the preservation and propagation of DNA as the genetic information in its original form, to guarantee stable inheritance through multiple generations. On the other hand, cells or organisms must be able to adapt to lasting environmental changes. The adaptive mechanisms can include qualitative or quantitative modifications of the genetic material. Qualitative modifications include DNA mutations, in which coding sequences are altered resulting in a structurally and/or functionally different protein. Gene amplification is a quantitative modification, whereby the actual number of complete coding sequence, i.e., a gene, increases, leading to an increased number of available templates for transcription, an increased number of translatable transcripts, and, ultimately, to an increased abundance of the protein encoded by the amplified gene.

The phenomenon of gene amplification and its underlying mechanisms have been investigated in vitro in several prokaryotic and eukaryotic culture systems. The best-characterized example of gene amplification involves the culture of eukaryotic cells in medium containing variable concentrations of the cytotoxic drug methotrexate (MTX). MTX is a folic acid analogue and interferes with DNA synthesis by blocking the enzyme dihydrofolate reductase (DHFR). During the initial exposure to low concentrations of MTX most cells (>99.9%) will die. A small number of cells survive, and are capable of growing in increasing concentrations of MTX by producing large amounts of DHFR-RNA and protein. The basis of this overproduction is the amplification of the single DHFR gene. The additional copies of the gene are found as extrachromosomal copies in the form of small, supernumerary chromosomes (double minutes) or as integrated chromosomal copies.

Gene amplification is most commonly encountered in the development of resistance to cytotoxic drugs (antibiotics for bacteria and chemotherapeutic agents for eukaryotic cells) and neoplastic transformation. Transformation of a eukaryotic cell as a spontaneous event or due to a viral or chemical/environmental insult is typically associated with changes in the genetic material of that cell. One of the most common genetic changes observed in human malignancies are mutations of the p53 protein. p53 controls the transition of cells from the stationary (G1) to the replicative (S) phase and prevents this transition in the presence of DNA damage. In other words, one of the main consequences of disabling p53 mutations is the accumulation and propagation of DNA damage, i.e., genetic changes. Common types of genetic changes in neoplastic cells are, in addition to point mutations, amplifications and gross, structural alterations, such as translocations.

The amplification of DNA sequences may indicate a specific functional requirement as illustrated in the DHFR experimental system. Therefore, the amplification of certain oncogenes in malignancies points toward a causative role of these genes in the process of malignant transformation and maintenance of the transformed phenotype. This hypothesis has gained support in recent studies. For example, the bcl-2 protein was found to be amplified in certain types of non-Hodgkin's lymphoma. This protein inhibits apoptosis and leads to the progressive accumulation of neoplastic cells. Members of the gene family of growth factor receptors have been found to be amplified in various types of cancers suggesting that overexpression of these receptors may make neoplastic cells less susceptible to limiting amounts of available growth factor. Examples include the amplification of the androgen receptor in recurrent prostate cancer during androgen deprivation therapy and the amplification of the growth factor receptor homologue ERB2 in breast cancer. Lastly, genes involved in intracellular signaling and control of cell cycle progression can undergo amplification during malignant transformation. This is illustrated by the amplification of the bcl-I and ras genes in various epithelial and lymphoid neoplasms.

These earlier studies illustrate the feasibility of identifying amplified DNA sequences in neoplasms, because this approach can identify genes important for malignant transformation. The case of ERB2 also demonstrates the feasibility from a therapeutic standpoint, since transforming proteins may represent novel and specific targets for tumor therapy.

Several different techniques can be used to demonstrate amplified genomic sequences. Classical cytogenetic analysis of chromosome spreads prepared from cancer cells is adequate to identify gross structural alterations, such as translocations, deletions and inversions. Amplified genomic regions can only be visualized, if they involve large regions with high copy numbers or are present as extrachromosomal material. While cytogenetics was the first technique to demonstrate the consistent association of specific chromosomal changes with particular neoplasms, it is inadequate for the identification and isolation of manageable DNA sequences. The more recently developed technique of comparative genomic hybridization (CGH) has illustrated the widespread phenomenon of genomic amplification in neoplasms. Tumor and normal DNA are hybridized simultaneously onto metaphases of normal cells and the entire genome can be screened by image analysis for DNA sequences that are present in the tumor at an increased frequency. (WO 93/18,186; Gray et al., Radiation Res. 137:275-289 [1994]). As a screening method, this type of analysis has revealed a large number of recurring amplicons (a stretch of amplified DNA) in a variety of human neoplasms. Although CGH is more sensitive than classical cytogenetic analysis in identifying amplified stretches of DNA, it does not allow a rapid identification and isolation of coding sequences within the amplicon by standard molecular genetic techniques.

The most sensitive methods to detect gene amplification are polymerase chain reaction (PCR)-based assays. These assays utilize very small amount of tumor DNA as starting material, are exquisitely sensitive, provide DNA that is amenable to further analysis, such as sequencing and are suitable for high-volume throughput analysis.

The above-mentioned assays are not mutually exclusive, but are frequently used in combination to identify amplifications in neoplasms. While cytogenetic analysis and CGH represent screening methods to survey the entire genome for amplified regions, PCR-based assays are most suitable for the final identification of coding sequences, i.e., genes in amplified regions.

According to the present invention, such genes can be identified by quantitative PCR (S. Gelmini et al., Clin. Chem., 43:752 [1997]), by comparing DNA from a variety of primary tumors, including breast, lung, colon, prostate, brain, liver, kidney, pancreas, spleen, thymus, testis, ovary, uterus, etc., preferably renal cell carcinoma, tumor, or tumor cell lines, with pooled DNA from healthy donors. Quantitative PCR is performed using a TaqMan instrument (ABI). Gene-specific primers and fluorogenic probes are designed based upon the coding sequences of the DNAs.

Human lung carcinoma cell lines include A549 (SRCC768), Calu-1 (SRCC769), Calu-6 (SRCC770), H157 (SRCC771), H441 (SRCC772), H460 (SRCC773), SKMES-1 (SRCC774), SW900 (SRCC775), H522 (SRCC832), and H810 (SRCC833), all available from ATCC. Primary human lung tumor cells usually derive from adenocarcinomas, squamous cell carcinomas, large cell carcinomas, non-small cell carcinomas, small cell carcinomas, and broncho alveolar carcinomas, and include, for example, SRCC724 (adenocarcinoma, abbreviated as “AdenoCa”)(LT1), SRCC725 (squamous cell carcinoma, abbreviated as “SqCCa)(LT1a), SRCC726 (adenocarcinoma)(LT2), SRCC727 (adenocarcinoma)(LT3), SRCC728 (adenocarcinoma)(LT4), SRCC729 (squamous cell carcinoma)(LT6), SRCC730 (adeno/squamous cell carcinoma)(LT7), SRCC731 (adenocarcinoma)(LT9), SRCC732 (squamous cell carcinoma)(LT10), SRCC733 (squamous cell carcinoma)(LT11), SRCC734 (adenocarcinoma)(LT12), SRCC735 (adeno/squamous cell carcinoma)(LT13), SRCC736 (squamous cell carcinoma)(LT15), SRCC737 (squamous cell carcinoma)(LT16), SRCC738 (squamous cell carcinoma)(LT17), SRCC739 (squamous cell carcinoma)(LT18), SRCC740 (squamous cell carcinoma)(LT19), SRCC741 (lung cell carcinoma, abbreviated as “LCCa”)(LT21), SRCC811 (adenocarcinoma)(LT22), SRCC825 (adenocarcinoma)(LT8), SRCC886 (adenocarcinoma)(LT25), SRCC887 (squamous cell carcinoma) (LT26), SRCC888 (adeno-BAC carcinoma) (LT27), SRCC889 (squamous cell carcinoma) (LT28), SRCC890 (squamous cell carcinoma) (LT29), SRCC891 (adenocarcinoma) (LT30), SRCC892 (squamous cell carcinoma) (LT31), SRCC894 (adenocarcinoma) (LT33). Also included are human lung tumors designated SRCC1125 [HF-000631], SRCC1127 [HF-000641], SRCC1129 [HF-000643], SRCC1133 [HF-000840], SRCC1135 [HF-000842], SRCC1227 [HF-001291], SRCC1229 [HF-001293], SRCC1230 [HF-001294], SRCC1231 [HF-001295], SRCC1232 [HF-001296], SRCC1233 [HF-001297], SRCC1235 [HF-001299], and SRCC1236 [HF-001300].

Colon cancer cell lines include, for example, ATCC cell lines SW480 (adenocarcinoma, SRCC776), SW620 (lymph node metastasis of colon adenocarcinoma, SRCC777), Colo320 (carcinoma, SRCC778), HT29 (adenocarcinoma, SRCC779), HM7 (a high mucin producing variant of ATCC colon adenocarcinoma cell line, SRCC780, obtained from Dr. Robert Warren, UCSF), CaWiDr (adenocarcinoma, SRCC781), HCT116 (carcinoma, SRCC782), SKCO1 (adenocarcinoma, SRCC783), SW403 (adenocarcinoma, SRCC784), LS174T (carcinoma, SRCC785), Colo205 (carcinoma, SRCC828), HCT15 (carcinoma, SRCC829), HCC2998 (carcinoma, SRCC830), and KM12 (carcinoma, SRCC831). Primary colon tumors include colon adenocarcinomas designated CT2 (SRCC742), CT3 (SRCC743), CT8 (SRCC744), CT10 (SRCC745), CT12 (SRCC746), CT14 (SRCC747), CT15 (SRCC748), CT16 (SRCC749), CT17 (SRCC750), CT1 (SRCC751), CT4 (SRCC752), CT5 (SRCC753), CT6 (SRCC754), CT7 (SRCC755), CT9 (SRCC756), CT11 (SRCC757), CT18 (SRCC758), CT19 (adenocarcinoma, SRCC906), CT20 (adenocarcinoma, SRCC907), CT21 (adenocarcinoma, SRCC908), CT22 (adenocarcinoma, SRCC909), CT23 (adenocarcinoma, SRCC910), CT24 (adenocarcinoma, SRCC911), CT25 (adenocarcinoma, SRCC912), CT26 (adenocarcinoma, SRCC913), CT27 (adenocarcinoma, SRCC914), CT28 (adenocarcinoma, SRCC915), CT29 (adenocarcinoma, SRCC916), CT30 (adenocarcinoma, SRCC917), CT31 (adenocarcinoma, SRCC918), CT32 (adenocarcinoma, SRCC919), CT33 (adenocarcinoma, SRCC920), CT35 (adenocarcinoma, SRCC921), and CT36 (adenocarcinoma, SRCC922). Also included are human colon tumor centers designated SRCC1051 [HF-000499], SRCC1052 [HF-000539], SRCC1053 [HF-000575], SRCC1054 [HF-000698], SRCC1142 [HF-000762], SRCC1144 [HF-000789], SRCC1146 [HF-000795] and SRCC1148[HF-000811].

Human breast carcinoma cell lines include, for example, HBL100 (SRCC759), MB435s (SRCC760), T47D (SRCC761), MB468(SRCC762), MB175 (SRCC763), MB361 (SRCC764), BT20 (SRCC765), MCF7 (SRCC766), and SKBR3 (SRCC767), and human breast tumor center designated SRCC1057 [HF-000545]. Also included are human breast tumors designated SRCC1094, SRCC1095, SRCC1096, SRCC1097, SRCC1098, SRCC1099, SRCC1100, SRCC1101, and human breast-met-lung-NS tumor designated SRCC893 [LT 32].

Human kidney tumor centers include SRCC989 [HF-000611] and SRCC1014 [HF-000613]. Human testis tumor center includes SRCC1001 [HF-000733] and testis tumor margin SRCC999 [HF-000716].

Human parathyroid tumor includes SRCC1002 [HF-000831] and SRCC1003 [HF-000832].

I. Tissue Distribution

The results of the gene amplification assays herein can be verified by further studies, such as, by determining mRNA expression in various human tissues.

As noted before, gene amplification and/or gene expression in various tissues may be measured by conventional Southern blotting, Northern blotting to quantitate the transcription of mRNA (Thomas, Proc. Natl. Acad. Sci. USA, 77:5201-5205 [1980]), dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes.

Gene expression in various tissues, alternatively, may be measured by immunological methods, such as immunohistochemical staining of tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide or against a synthetic peptide based on the DNA sequences provided herein or against exogenous sequence fused to sequence CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 DNA and encoding a specific antibody epitope. General techniques for generating antibodies, and special protocols for Northern blotting and in situ hybridization are provided hereinbelow.

J. Chromosome Mapping

If the amplification of a given gene is functionally relevant, then that gene should be amplified more than neighboring genomic regions which are not important for tumor survival. To test this, the gene can be mapped to a particular chromosome, e.g., by radiation-hybrid analysis. The amplification level is then determined at the location identified, and at the neighboring genomic region. Selective or preferential amplification at the genomic region to which the gene has been mapped is consistent with the possibility that the gene amplification observed promotes tumor growth or survival. Chromosome mapping includes both framework and epicenter mapping. For further details see, e.g., Stewart et al., Genome Research, 7:422-433 (1997).

K. Antibody Binding Studies

The results of the gene amplification study can be further verified by antibody binding studies, in which the ability of anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibodies to inhibit the expression of CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptides on tumor (cancer) cells is tested. Exemplary antibodies include polyclonal, monoclonal, humanized, bispecific, and heteroconjugate antibodies, the preparation of which will be described hereinbelow.

Antibody binding studies may be carried out in any known assay method, such as competitive binding assays, direct and indirect sandwich assays, and immunoprecipitation assays. Zola, Monoclonal Antibodies: A Manual of Techniques, pp. 147-158 (CRC Press, Inc., 1987).

Competitive binding assays rely on the ability of a labeled standard to compete with the test sample analyte for binding with a limited amount of antibody. The amount of target protein (encoded by a gene amplified in a tumor cell) in the test sample is inversely proportional to the amount of standard that becomes bound to the antibodies. To facilitate determining the amount of standard that becomes bound, the antibodies preferably are insolubilized before or after the competition, so that the standard and analyte that are bound to the antibodies may conveniently be separated from the standard and analyte which remain unbound.

Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

For immunohistochemistry, the tumor sample may be fresh or frozen or may be embedded in paraffin and fixed with a preservative such as formalin, for example.

L. Cell-Based Tumor Assays

Cell-based assays and animal models for tumors (e.g., cancers) can be used to verify the findings of the gene amplification assay, and further understand the relationship between the genes identified herein and the development and pathogenesis of neoplastic cell growth. The role of gene products identified herein in the development and pathology of tumor or cancer can be tested by using primary tumor cells or cells lines that have been identified to amplify the genes herein. Such cells include, for example, the breast, colon and lung cancer cells and cell lines listed above.

In a different approach, cells of a cell type known to be involved in a particular tumor are transfected with the cDNAs herein, and the ability of these cDNAs to induce excessive growth is analyzed. Suitable cells include, for example, stable tumor cells lines such as, the B104-1-1 cell line (stable NIH-3T3 cell line transfected with the neu protooncogene) and ras-transfected NIH-3T3 cells, which can be transfected with the desired gene, and monitored for tumorogenic growth. Such transfected cell lines can then be used to test the ability of poly- or monoclonal antibodies or antibody compositions to inhibit tumorogenic cell growth by exerting cytostatic or cytotoxic activity on the growth of the transformed cells, or by mediating antibody-dependent cellular cytotoxicity (ADCC). Cells transfected with the coding sequences of the genes identified herein can further be used to identify drug candidates for the treatment of cancer.

In addition, primary cultures derived from tumors in transgenic animals (as described below) can be used in the cell-based assays herein, although stable cell lines are preferred. Techniques to derive continuous cell lines from transgenic animals are well known in the art (see, e.g., Small et al., Mol. Cell. Biol., 5:642-648 [1985]).

M. Animal Models

A variety of well known animal models can be used to further understand the role of the genes identified herein in the development and pathogenesis of tumors, and to test the efficacy of candidate therapeutic agents, including antibodies, and other antagonists of the native polypeptides, including small molecule antagonists. The in vivo nature of such models makes them particularly predictive of responses in human patients. Animal models of tumors and cancers (e.g., breast cancer, colon cancer, prostate cancer, lung cancer, etc.) include both non-recombinant and recombinant (transgenic) animals. Non-recombinant animal models include, for example, rodent, e.g., murine models. Such models can be generated by introducing tumor cells into syngeneic mice using standard techniques, e.g., subcutaneous injection, tail vein injection, spleen implantation, intraperitoneal implantation, implantation under the renal capsule, or orthopin implantation, e.g., colon cancer cells implanted in colonic tissue. (See, e.g., PCT publication No. WO 97/33551, published Sep. 18, 1997).

Probably the most often used animal species in oncological studies are immunodeficient mice and, in particular, nude mice. The observation that the nude mouse with hypo/aplasia could successfully act as a host for human tumor xenografts has lead to its widespread use for this purpose. The autosomal recessive nu gene has been introduced into a very large number of distinct congenic strains of nude mouse, including, for example, ASW, A/He, AKR, BALB/c, B10.LP, C17, C3H, C57BL, C57, CBA, DBA, DDD, I/st, NC, NFR, NFS, NFS/N, NZB, NZC, NZW, P, RIII and SJL. In addition, a wide variety of other animals with inherited immunological defects other than the nude mouse have been bred and used as recipients of tumor xenografts. For further details see, e.g., The Nude Mouse in Oncology Research, E. Boven and B. Winograd, eds., CRC Press, Inc., 1991.

The cells introduced into such animals can be derived from known tumor/cancer cell lines, such as, any of the above-listed tumor cell lines, and, for example, the B104-1-1 cell line (stable NIH-3T3 cell line transfected with the neu protooncogene); ras-transfected NIH-3T3 cells; Caco-2 (ATCC HTB-37); a moderately well-differentiated grade II human colon adenocarcinoma cell line, HT-29 (ATCC HTB-38), or from tumors and cancers. Samples of tumor or cancer cells can be obtained from patients undergoing surgery, using standard conditions, involving freezing and storing in liquid nitrogen (Karmali et al., Br. J. Cancer, 48:689-696 [1983]).

Tumor cells can be introduced into animals, such as nude mice, by a variety of procedures. The subcutaneous (s.c.) space in mice is very suitable for tumor implantation. Tumors can be transplanted s.c. as solid blocks, as needle biopsies by use of a trochar, or as cell suspensions. For solid block or trochar implantation, tumor tissue fragments of suitable size are introduced into the s.c. space. Cell suspensions are freshly prepared from primary tumors or stable tumor cell lines, and injected subcutaneously. Tumor cells can also be injected as subdermal implants. In this location, the inoculum is deposited between the lower part of the dermal connective tissue and the s.c. tissue. Boven and Winograd (1991), supra.

Animal models of breast cancer can be generated, for example, by implanting rat neuroblastoma cells (from which the neu oncogen was initially isolated), or neu-transformed NIH-3T3 cells into nude mice, essentially as described by Drebin et al., PNAS USA, 83:9129-9133 (1986).

Similarly, animal models of colon cancer can be generated by passaging colon cancer cells in animals, e.g., nude mice, leading to the appearance of tumors in these animals. An orthotopic transplant model of human colon cancer in nude mice has been described, for example, by Wang et al., Cancer Research, 54:4726-4728 (1994) and Too et al., Cancer Research, 55:681-684 (1995). This model is based on the so-called “METAMOUSE” sold by AntiCancer, Inc., (San Diego, Calif.).

Tumors that arise in animals can be removed and cultured in vitro. Cells from the in vitro cultures can then be passaged to animals. Such tumors can serve as targets for further testing or drug screening. Alternatively, the tumors resulting from the passage can be isolated and RNA from pre-passage cells and cells isolated after one or more rounds of passage analyzed for differential expression of genes of interest. Such passaging techniques can be performed with any known tumor or cancer cell lines.

For example, Meth A, CMS4, CMS5, CMS21, and WEHI-164 are chemically induced fibrosarcomas of BALB/c female mice (DeLeo et al., J. Exp. Med., 146:720 [1977]), which provide a highly controllable model system for studying the anti-tumor activities of various agents (Palladino et al., J. Immunol. 138:4023-4032 [1987]). Briefly, tumor cells are propagated in vitro in cell culture. Prior to injection into the animals, the cell lines are washed and suspended in buffer, at a cell density of about 10×10⁶ to 10×10⁷ cells/ml. The animals are then infected subcutaneously with 10 to 100 μl of the cell suspension, allowing one to three weeks for a tumor to appear.

In addition, the Lewis lung (3LL) carcinoma of mice, which is one of the most thoroughly studied experimental tumors, can be used as an investigational tumor model. Efficacy in this tumor model has been correlated with beneficial effects in the treatment of human patients diagnosed with small cell carcinoma of the lung (SCCL). This tumor can be introduced in normal mice upon injection of tumor fragments from an affected mouse or of cells maintained in culture (Zupi et al., Br. J. Cancer, 41: suppl. 4:309 [1980]), and evidence indicates that tumors can be started from injection of even a single cell and that a very high proportion of infected tumor cells survive. For further information about this tumor model see, Zacharski, Haemostasis, 16:300-320 [1986]).

One way of evaluating the efficacy of a test compound in an animal model on an implanted tumor is to measure the size of the tumor before and after treatment. Traditionally, the size of implanted tumors has been measured with a slide caliper in two or three dimensions. The measure limited to two dimensions does not accurately reflect the size of the tumor, therefore, it is usually converted into the corresponding volume by using a mathematical formula. However, the measurement of tumor size is very inaccurate. The therapeutic effects of a drug candidate can be better described as treatment-induced growth delay and specific growth delay. Another important variable in the description of tumor growth is the tumor volume doubling time. Computer programs for the calculation and description of tumor growth are also available, such as the program reported by Rygaard and Spang-Thomsen, Proc. 6th Int. Workshop on Immune-Deficient Animals, Wu and Sheng eds., Basel, 1989, 301. It is noted, however, that necrosis and inflammatory responses following treatment may actually result in an increase in tumor size, at least initially. Therefore, these changes need to be carefully monitored, by a combination of a morphometric method and flow cytometric analysis.

Recombinant (transgenic) animal models can be engineered by introducing the coding portion of the genes identified herein into the genome of animals of interest, using standard techniques for producing transgenic animals. Animals that can serve as a target for transgenic manipulation include, without limitation, mice, rats, rabbits, guinea pigs, sheep, goats, pigs, and non-human primates, e.g., baboons, chimpanzees and monkeys. Techniques known in the art to introduce a transgene into such animals include pronucleic microinjection (Hoppe and Wanger, U.S. Pat. No. 4,873,191); retrovirus-mediated gene transfer into germ lines (e.g., Van der Putten et al., Proc. Natl. Acad. Sci. USA, 82:6148-615 [1985]); gene targeting in embryonic stem cells (Thompson et al., Cell, 56:313-321 [1989]); electroporation of embryos (Lo, Mol. Cell Biol., 3:1803-1814 [1983]); sperm-mediated gene transfer (Lavitrano et al., Cell, 57:717-73 [1989]). For review, see, for example, U.S. Pat. No. 4,736,866.

For the purpose of the present invention, transgenic animals include those that carry the transgene only in part of their cells (“mosaic animals”). The transgene can be integrated either as a single transgene, or in concatamers, e.g., head-to-head or head-to-tail tandems. Selective introduction of a transgene into a particular cell type is also possible by following, for example, the technique of Lasko et al., Proc. Natl. Acad. Sci. USA, 89:6232-636 (1992).

The expression of the transgene in transgenic animals can be monitored by standard techniques. For example, Southern blot analysis or PCR amplification can be used to verify the integration of the transgene. The level of mRNA expression can then be analyzed using techniques such as in situ hybridization, Northern blot analysis, PCR, or immunocytochemistry. The animals are further examined for signs of tumor or cancer development.

Alternatively, “knock out” animals can be constructed which have a defective or altered gene encoding a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide identified herein, as a result of homologous recombination between the endogenous gene encoding the polypeptide and altered genomic DNA encoding the same polypeptide introduced into an embryonic cell of the animal. For example, cDNA encoding a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide can be used to clone genomic DNA encoding that polypeptide in accordance with established techniques. A portion of the genomic DNA encoding a particular CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide can be deleted or replaced with another gene, such as a gene encoding a selectable marker which can be used to monitor integration. Typically, several kilobases of unaltered flanking DNA (both at the 5′ and 3′ ends) are included in the vector [see, e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see, e.g., Li et al., Cell, 69:915 (1992)]. The selected cells are then injected into a blastocyst of an animal (e.g., a mouse or rat) to form aggregation chimeras [see, e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, by their ability to defend against certain pathological conditions and by their development of pathological conditions due to absence of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide.

The efficacy of antibodies specifically binding the polypeptides identified herein and other drug candidates, can be tested also in the treatment of spontaneous animal tumors. A suitable target for such studies is the feline oral squamous cell carcinoma (SCC). Feline oral SCC is a highly invasive, malignant tumor that is the most common oral malignancy of cats, accounting for over 60% of the oral tumors reported in this species. It rarely metastasizes to distant sites, although this low incidence of metastasis may merely be a reflection of the short survival times for cats with this tumor. These tumors are usually not amenable to surgery, primarily because of the anatomy of the feline oral cavity. At present, there is no effective treatment for this tumor. Prior to entry into the study, each cat undergoes complete clinical examination, biopsy, and is scanned by computed tomography (CT). Cats diagnosed with sublingual oral squamous cell tumors are excluded from the study. The tongue can become paralyzed as a result of such tumor, and even if the treatment kills the tumor, the animals may not be able to feed themselves. Each cat is treated repeatedly, over a longer period of time. Photographs of the tumors will be taken daily during the treatment period, and at each subsequent recheck. After treatment, each cat undergoes another CT scan. CT scans and thoracic radiograms are evaluated every 8 weeks thereafter. The data are evaluated for differences in survival, response and toxicity as compared to control groups. Positive response may require evidence of tumor regression, preferably with improvement of quality of life and/or increased life span.

In addition, other spontaneous animal tumors, such as fibrosarcoma, adenocarcinoma, lymphoma, chrondroma, leiomyosarcoma of dogs, cats, and baboons can also be tested. Of these mammary adenocarcinoma in dogs and cats is a preferred model as its appearance and behavior are very similar to those in humans. However, the use of this model is limited by the rare occurrence of this type of tumor in animals.

N. Screening Assays for Drug Candidates

Screening assays for drug candidates are designed to identify compounds that bind or complex with the polypeptides encoded by the genes identified herein, or otherwise interfere with the interaction of the encoded polypeptides with other cellular proteins. Such screening assays will include assays amenable to high-throughput screening of chemical libraries, making them particularly suitable for identifying small molecule drug candidates. Small molecules contemplated include synthetic organic or inorganic compounds, including peptides, preferably soluble peptides, (poly)peptide-immunoglobulin fusions, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. The assays can be performed in a variety of formats, including protein-protein binding assays, biochemical screening assays, immunoassays and cell based assays, which are well characterized in the art.

All assays are common in that they call for contacting the drug candidate with a polypeptide encoded by a nucleic acid identified herein under conditions and for a time sufficient to allow these two components to interact.

In binding assays, the interaction is binding and the complex formed can be isolated or detected in the reaction mixture. In a particular embodiment, the polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the polypeptide and drying. Alternatively, an immobilized antibody, e.g., a monoclonal antibody, specific for the polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide encoded by a gene identified herein, its interaction with that polypeptide can be assayed by methods well known for detecting protein-protein interactions. Such assays include traditional approaches, such as, cross-linking, co-immunoprecipitation, and co-purification through gradients or chromatographic columns. In addition, protein-protein interactions can be monitored by using a yeast-based genetic system described by Fields and co-workers [Fields and Song, Nature, 340:245-246 (1989); Chien et al., Proc. Natl. Acad. Sci. USA, 88: 9578-9582(1991)] as disclosed by Chevray and Nathans, Proc. Natl. Acad. Sci. USA, 89:5789-5793 (1991)]. Many transcriptional activators, such as yeast GAL4, consist of two physically discrete modular domains, one acting as the DNA-binding domain, while the other one functioning as the transcription activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36-encoding gene identified herein and other intra- or extracellular components can be tested as follows: usually a reaction mixture is prepared containing the product of the amplified gene and the intra- or extracellular component under conditions and for a time allowing for the interaction and binding of the two products. To test the ability of a test compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

To assay for antagonists, the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide may be added to a cell along with the compound to be screened for a particular activity and the ability of the compound to inhibit the activity of interest in the presence of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide indicates that the compound is an antagonist to the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide. Alternatively, antagonists may be detected by combining the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide and a potential antagonist with membrane-bound CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide receptors or recombinant receptors under appropriate conditions for a competitive inhibition assay. The CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide can be labeled, such as by radioactivity, such that the number of CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide molecules bound to the receptor can be used to determine the effectiveness of the potential antagonist. The gene encoding the receptor can be identified by numerous methods known to those of skill in the art, for example, ligand panning and FACS sorting. Coligan et al., Current Protocols in Immun., 1(2): Chapter 5 (1991). Preferably, expression cloning is employed wherein polyadenylated RNA is prepared from a cell responsive to the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide and a cDNA library created from this RNA is divided into pools and used to transfect COS cells or other cells that are not responsive to the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide. Transfected cells that are grown on glass slides are exposed to labeled CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide. The CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide can be labeled by a variety of means including iodination or inclusion of a recognition site for a site-specific protein kinase. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor.

As an alternative approach for receptor identification, labeled CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide can be photoaffinity-linked with cell membrane or extract preparations that express the receptor molecule. Cross-linked material is resolved by PAGE and exposed to X-ray film. The labeled complex containing the receptor can be excised, resolved into peptide fragments, and subjected to protein micro-sequencing. The amino acid sequence obtained from micro-sequencing would be used to design a set of degenerate oligonucleotide probes to screen a cDNA library to identify the gene encoding the putative receptor.

In another assay for antagonists, mammalian cells or a membrane preparation expressing the receptor would be incubated with labeled CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide in the presence of the candidate compound. The ability of the compound to enhance or block this interaction could then be measured.

More specific examples of potential antagonists include an oligonucleotide that binds to the fusions of immunoglobulin with the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, a potential antagonist may be a closely related protein, for example, a mutated form of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide that recognizes the receptor but imparts no effect, thereby competitively inhibiting the action of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide.

Another potential CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide antagonist is an antisense RNA or DNA construct prepared using antisense technology, where, e.g., an antisense RNA or DNA molecule acts to block directly the translation of mRNA by hybridizing to targeted mRNA and preventing protein translation. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide herein, is used to design an antisense RNA oligonucleotide of from about 10 to 40 base pairs in length. A DNA oligonucleotide is designed to be complementary to a region of the gene involved in transcription (triple helix—see, Lee et al., Nucl. Acids Res., 6:3073 (1979); Cooney et al., Science, 241: 456 (1988); Dervan et al., Science, 251:1360 (1991)), thereby preventing transcription and the production of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide. The antisense RNA oligonucleotide hybridizes to the mRNA in vivo and blocks translation of the mRNA molecule into the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide (antisense#Okano, Neurochem. 56:560 (1991); Oligodeoxynucleotides as Antisense Inhibitors of Gene Expression (CRC Press: Boca Raton, Fla., 1988). The oligonucleotides described above can also be delivered to cells such that the antisense RNA or DNA may be expressed in vivo to inhibit production of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide. When antisense DNA is used, oligodeoxyribonucleotides derived from the translation-initiation site, e.g., between about -10 and +10 positions of the target gene nucleotide sequence, are preferred.

Antisense RNA or DNA molecules are generally at least about 5 bases in length, about 10 bases in length, about 15 bases in length, about 20 bases in length, about 25 bases in length, about 30 bases in length, about 35 bases in length, about 40 bases in length, about 45 bases in length, about 50 bases in length, about 55 bases in length, about 60 bases in length, about 65 bases in length, about 70 bases in length, about 75 bases in length, about 80 bases in length, about 85 bases in length, about 90 bases in length, about 95 bases in length, about 100 bases in length, or more.

Potential antagonists include small molecules that bind to the active site, the receptor binding site, or growth factor or other relevant binding site of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, thereby blocking the normal biological activity of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37 ; Ephrin A1; Laminin beta 2; Integrin alpha 1; Stanniocalcin 1; Thrombospondin 4; or CD36 polypeptide. Examples of small molecules include, but are not limited to, small peptides or peptide-like molecules, preferably soluble peptides, and synthetic non-peptidyl organic or inorganic compounds.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology, 4:469-471 (1994), and PCT publication No. WO 97/33551 (published Sep. 18, 1997).

Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex. For further details see, e.g., PCT publication No. WO 97/33551, supra.

These small molecules can be identified by any one or more of the screening assays discussed hereinabove and/or by any other screening techniques well known for those skilled in the art.

O. Compositions and Methods for the Treatment of Tumors

The compositions useful in the treatment of tumors associated with the amplification of the genes identified herein include, without limitation, antibodies, small organic and inorganic molecules, peptides, phosphopeptides, antisense and ribozyme molecules, triple helix molecules, etc., that inhibit the expression and/or activity of the target gene product.

For example, antisense RNA and RNA molecules act to directly block the translation of mRNA by hybridizing to targeted mRNA and preventing protein translation. When antisense DNA is used, oligodeoxyribonucleotides derived from the translation initiation site, e.g., between about -10 and +10 positions of the target gene nucleotide sequence, are preferred.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology, 4:469-471 (1994), and PCT publication No. WO 97/33551 (published Sep. 18, 1997).

Nucleic acid molecules in triple helix formation used to inhibit transcription should be single-stranded and composed of deoxynucleotides. The base composition of these oligonucleotides is designed such that it promotes triple helix formation via Hoogsteen base pairing rules, which generally require sizeable stretches of purines or pyrimidines on one strand of a duplex. For further details see, e.g., PCT publication No. WO 97/33551, supra.

These molecules can be identified by any or any combination of the screening assays discussed hereinabove and/or by any other screening techniques well known for those skilled in the art.

P. Antibodies

Some of the most promising drug candidates according to the present invention are antibodies and antibody fragments which may inhibit the production or the gene product of the amplified genes identified herein and/or reduce the activity of the gene products.

1. Polyclonal Antibodies

Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

2. Monoclonal Antibodies

The anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, including fragments, or a fusion protein of such protein or a fragment thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection (ATCC), Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison et al., supra] or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

3. Human and Humanized Antibodies

The anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibodies may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol. 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from anon-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al., and Boerner et al., are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology, 10:779-783 (1992); Lonberg et al., Nature, 368:856-859 (1994); Morrison, Nature, 368:812-13 (1994); Fishwild et al., Nature Biotechnology, 14:845-51 (1996); Neuberger, Nature Biotechnology, 14:826 (1996); Lonberg and Huszar, Intern. Rev. Immunol., 13:65-93 (1995).

4. Antibody Dependent Enzyme Mediated Prodrug Therapy (ADEPT)

The antibodies of the present invention may also be used in ADEPT by conjugating the antibody to a prodrug-activating enzyme which converts a prodrug (e.g., a peptidyl chemotherapeutic agent, see WO 81/01145) to an active anti-cancer drug. See, for example, WO 88/07378 and U.S. Pat. No. 4,975,278.

The enzyme component of the immunoconjugate useful for ADEPT includes any enzyme capable of acting on a prodrug in such as way so as to convert it into its more active, cytotoxic form.

Enzymes that are useful in the method of this invention include, but are not limited to, glycosidase, glucose oxidase, human lysosyme, human glucuronidase, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug 5-fluorouracil; proteases, such as serratiaprotease, thermolysin, subtilisin, carboxypeptidases (e.g., carboxypeptidase G2 and carboxypeptidase A) and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin Vamidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes” can be used to convert the prodrugs of the invention into free active drugs (see, e.g., Massey, Nature, 328:457-458 (1987)). Antibody-abzyme conjugates can be prepared as described herein for delivery of the abzyme to a tumor cell population.

The enzymes of this invention can be covalently bound to the anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibodies by techniques well known in the art such as the use of the heterobifunctional cross-linking agents discussed above. Alternatively, fusion proteins comprising at least the antigen binding region of the antibody of the invention linked to at least a functionally active portion of an enzyme of the invention can be constructed using recombinant DNA techniques well known in the art (see, e.g., Neuberger et al., Nature, 312:604-608 (1984)).

5. Bispecific Antibodies

Bispecific antibodies are monoclonal, preferably human or humanized, antibodies that have binding specificities for at least two different antigens. In the present case, one of the binding specificities is for the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 [1983]). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies can be prepared as full length antibodies or antibody fragments (e.g., F(ab′)₂ bispecific antibodies). Techniques for generating bispecific antibodies from antibody fragments have been described in the literature. For example, bispecific antibodies can be prepared using chemical linkage. Brennan et al., Science, 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med., 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol., 152:5368 (1994).

Antibodies with more than two valencies are contemplated. For example, trispecific antibodies can be prepared. Tutt et al., J. Immunol., 147:60 (1991).

Exemplary bispecific antibodies may bind to two different epitopes on a given polypeptide herein. Alternatively, an anti-polypeptide arm may be combined with an arm which binds to a triggering molecule on a leukocyte such as a T-cell receptor molecule (e.g., CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular polypeptide. Bispecific antibodies may also be used to localize cytotoxic agents to cells which express a particular polypeptide. These antibodies possess a polypeptide-binding arm and an arm which binds a cytotoxic agent or a radionuclide chelator, such as EOTUBE, DPTA, DOTA, or TETA. Another bispecific antibody of interest binds the polypeptide and further binds tissue factor (TF).

6. Heteroconjugate Antibodies

Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

7. Effector Function Engineering

It may be desirable to modify the antibody of the invention with respect to effector function, so as to enhance the effectiveness of the antibody in treating cancer, for example. For example, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See, Caron et al., J. Exp Med., 176:1191-1195 (1992) and Shopes, J. Immunol., 148:2918-2922 (1992). Homodimeric antibodies with enhanced anti-tumor activity may also be prepared using heterobifunctional cross-linkers as described in Wolff et al., Cancer Research, 53:2560-2565 (1993). Alternatively, an antibody can be engineered which has dual Fc regions and may thereby have enhanced complement lysis and ADCC capabilities. See, Stevenson et al., Anti-Cancer Drug Design, 3:219-230 (1989).

8. Immunoconjugates

The invention also pertains to immunoconjugates comprising an antibody conjugated to a cytotoxic agent such as a chemotherapeutic agent, toxin (e.g., an enzymatically active toxin of bacterial, fungal, plant or animal origin, or fragments thereof, or a small molecule toxin), or a radioactive isotope (i.e., a radioconjugate).

Chemotherapeutic agents useful in the generation of such immunoconjugates have been described above. Enzymatically active protein toxins and fragments thereof which can be used include diphtheria A chain, nonbinding active fragments of diphtheria toxin, cholera toxin, botulinus toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, saporin, mitogellin, restrictocin, phenomycin, enomycin and the tricothecenes. Small molecule toxins include, for example, calicheamicins, maytansinoids, palytoxin and CC1065. A variety of radionuclides are available for the production of radioconjugated antibodies. Examples include ²¹²Bi, ¹³¹I, ¹³¹In, ⁹⁰Y and ¹⁸⁶Re.

Conjugates of the antibody and cytotoxic agent are made using a variety of bifunctional protein coupling agents such as N-succinimidyl-3-(2-pyridyldithiol)propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See, WO94/11026.

In another embodiment, the antibody maybe conjugated to a “receptor” (such as streptavidin) for utilization in tumor pretargeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

The antibodies disclosed herein may also be formulated as immunoliposomes. Liposomes containing the antibody are prepared by methods known in the art, such as described in Epstein et al., Proc. Natl. Acad. Sci. USA, 82:3688 (1985); Hwang et al., Proc. Natl. Acad. Sci. USA, 77:4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257:286-288 (1982) via a disulfide interchange reaction. A chemotherapeutic agent (such as Doxorubicin) is optionally contained within the liposome. See, Gabizon et al., J. National Cancer Inst., 81 (19):1484 (1989).

Q. Pharmaceutical Compositions

Antibodies specifically binding the product of an amplified gene identified herein, as well as other molecules identified by the screening assays disclosed hereinbefore, can be administered for the treatment of tumors, including cancers, in the form of pharmaceutical compositions.

If the protein encoded by the amplified gene is intracellular and whole antibodies are used as inhibitors, internalizing antibodies are preferred. However, lipofections or liposomes can also be used to deliver the antibody, or an antibody fragment, into cells. Where antibody fragments are used, the smallest inhibitory fragment which specifically binds to the binding domain of the target protein is preferred. For example, based upon the variable region sequences of an antibody, peptide molecules can be designed which retain the ability to bind the target protein sequence. Such peptides can be synthesized chemically and/or produced by recombinant DNA technology (see, e.g., Marasco et al., Proc. Natl. Acad. Sci. USA, 90:7889-7893 [1993]).

Therapeutic formulations of the antibody are prepared for storage by mixing the antibody having the desired degree of purity with optional pharmaceutically acceptable carriers, excipients or stabilizers (Remington's Pharmaceutical Sciences, 16th edition, Osol, A. ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammoniumchloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, orimmunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Non-antibody compounds identified by the screening assays of the present invention can be formulated in an analogous manner, using standard techniques well known in the art.

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. ed. (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S#S bond formation through thio-disulfide interchange, stabilization may be achieved by modifying sulfhydryl residues, lyophilizing from acidic solutions, controlling moisture content, using appropriate additives, and developing specific polymer matrix compositions.

R. Methods of Treatment

It is contemplated that the antibodies and other anti-tumor compounds of the present invention may be used to treat various conditions, including those characterized by overexpression and/or activation of the amplified genes identified herein. Exemplary conditions or disorders to be treated with such antibodies and other compounds, including, but not limited to, small organic and inorganic molecules, peptides, antisense molecules, etc., include benign or malignant tumors (e.g., renal, liver, kidney, bladder, breast, gastric, ovarian, colorectal, prostate, pancreatic, lung, vulval, thyroid, hepatic carcinomas; sarcomas; glioblastomas; and various head and neck tumors); leukemias and lymphoid malignancies; other disorders such as neuronal, glial, astrocytal, hypothalamic and other glandular, macrophagal, epithelial, stromal and blastocoelic disorders; and inflammatory, angiogenic and immunologic disorders.

The anti-tumor agents of the present invention, e.g., antibodies, are administered to a mammal, preferably a human, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Intravenous administration of the antibody is preferred.

Other therapeutic regimens may be combined with the administration of the anti-cancer agents, e.g., antibodies of the instant invention. For example, the patient to be treated with such anti-cancer agents may also receive radiation therapy. Alternatively, or in addition, a chemotherapeutic agent may be administered to the patient. Preparation and dosing schedules for such chemotherapeutic agents may be used according to manufacturers′ instructions or as determined empirically by the skilled practitioner. Preparation and dosing schedules for such chemotherapy are also described in Chemotherapy Service Ed., M. C. Perry, Williams & Wilkins, Baltimore, Md. (1992). The chemotherapeutic agent may precede, or follow administration of the anti-tumor agent, e.g., antibody, or may be given simultaneously therewith. The antibody may be combined with an anti-oestrogen compound such as tamoxifen or an anti-progesterone such as onapristone (see, EP 616812) in dosages known for such molecules.

It may be desirable to also administer antibodies against other tumor associated antigens, such as antibodies which bind to the ErbB2, EGFR, ErbB3, ErbB4, or vascular endothelial factor (VEGF). Alternatively, or in addition, two or more antibodies binding the same or two or more different antigens disclosed herein may be co-administered to the patient. Sometimes, it may be beneficial to also administer one or more cytokines to the patient. In a preferred embodiment, the antibodies herein are co-administered with a growth inhibitory agent. For example, the growth inhibitory agent may be administered first, followed by an antibody of the present invention. However, simultaneous administration or administration of the antibody of the present invention first is also contemplated. Suitable dosages for the growth inhibitory agent are those presently used and may be lowered due to the combined action (synergy) of the growth inhibitory agent and the antibody herein.

For the prevention or treatment of disease, the appropriate dosage of an anti-tumor agent, e.g., an antibody herein will depend on the type of disease to be treated, as defined above, the severity and course of the disease, whether the agent is administered for preventive or therapeutic purposes, previous therapy, the patient's clinical history and response to the agent, and the discretion of the attending physician. The agent is suitably administered to the patient at one time or over a series of treatments.

For example, depending on the type and severity of the disease, about 1 μg/kg to 15 mg/kg (e.g., 0.1-20 mg/kg) of antibody is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

S. Articles of Manufacture

In another embodiment of the invention, an article of manufacture containing materials useful for the diagnosis or treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for diagnosing or treating the condition and may have a sterile access port (for example the container may be an intravenous solution bag or a vial having a stopper pierceable by a hypodermic injection needle). The active agent in the composition is usually an anti-tumor agent capable of interfering with the activity of a gene product identified herein, e.g., an antibody. The label on, or associated with, the container indicates that the composition is used for diagnosing or treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

T. Diagnosis and Prognosis of Tumors

While cell surface proteins, such as growth receptors overexpressed in certain tumors are excellent targets for drug candidates or tumor (e.g., cancer) treatment, the same proteins along with secreted proteins encoded by the genes amplified in tumor cells find additional use in the diagnosis and prognosis of tumors. For example, antibodies directed against the protein products of genes amplified in tumor cells can be used as tumor diagnostics or prognostics.

For example, antibodies, including antibody fragments, can be used to qualitatively or quantitatively detect the expression of proteins encoded by the amplified genes (“marker gene products”). The antibody preferably is equipped with a detectable, e.g., fluorescent label, and binding can be monitored by light microscopy, flow cytometry, fluorimetry, or other techniques known in the art. These techniques are particularly suitable, if the amplified gene encodes a cell surface protein, e.g., a growth factor. Such binding assays are performed essentially as described in section 5 above.

In situ detection of antibody binding to the marker gene products can be performed, for example, by immunofluorescence or immunoelectron microscopy. For this purpose, a histological specimen is removed from the patient, and a labeled antibody is applied to it, preferably by overlaying the antibody on a biological sample. This procedure also allows for determining the distribution of the marker gene product in the tissue examined. It will be apparent for those skilled in the art that a wide variety of histological methods are readily available for in situ detection.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, 10801 University Blvd., Manassas, Va. 20110-2209. All original deposits referred to in the present application were made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit. The deposit will be made available by ATCC under the terms of the Budapest Treaty, and subject to an agreement between Genentech, Inc., and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC § 122 and the Commissioner's rules pursuant thereto (including 37 CFR § 1.14 with particular reference to 886 OG 638).

Unless otherwise noted, the present invention uses standard procedures of recombinant DNA technology, such as those described hereinabove and in the following textbooks: Sambrook et al., Molecular Cloning: A Laboratory Manual, Cold Spring Harbor Press N.Y., 1989; Ausubel et al., Current Protocols in Molecular Biology, Green Publishing Associates and Wiley Interscience, N.Y., 1989; Innis et al., PCR Protocols: A Guide to Methods and Applications, Academic Press, Inc., N.Y., 1990; Harlow et al., Antibodies: A Laboratory Manual, Cold Spring Harbor Press, Cold Spring Harbor, 1988; Gait, Oligonucleotide Synthesis, IRL Press, Oxford, 1984; R. I. Freshney, Animal Cell Culture, 1987; Coligan et al., Current Protocols in Immunology, 1991.

### Example 1

Gene Expression Profiling in Silico: Relative Expression of Genes in Renal Cell Carcinoma Tissue Expression Profiling Using GeneExpress®

A proprietary database containing gene expression information (GeneExpress®, Gene Logic Inc., Gaithersburg, Md.) was analyzed in an attempt to identify polypeptides (and their encoding nucleic acids) whose expression is significantly upregulated in a particular tumor tissue(s) of interest as compared to other tumor(s) and/or normal tissues. Specifically, analysis of the GeneExpress® database was conducted using either software available through Gene Logic Inc., Gaithersburg, Md., for use with the GeneExpress® database or with proprietary software written and developed at Genentech, Inc. for use with the GeneExpress® database. The rating of positive hits in the analysis is based upon several criteria including, for example, tissue specificity, tumor specificity and expression level in normal essential and/or normal proliferating tissues. The following is a list of molecules whose tissue expression profile as determined from an analysis of the GeneExpress® database evidences high tissue expression and significant upregulation of expression in a specific tumor or tumors as compared to other tumor(s) and/or normal tissues and optionally relatively low expression in normal essential and/or normal proliferating tissues. As such, the molecules listed in Table 3 are excellent polypeptide targets for the diagnosis and therapy of cancer, such as renal cell carcinoma in mammals. Gene expression in renal cell carcinoma (RCC) relative to normal renal tissue is disclosed herein.

Twenty-two genes were identified that demonstrated a greater than 1.5-fold median tumor expression-to-normal expression ratio (Table 3). The median value was used to determine the ratio of expression because the median, rather than the average value, is not skewed by a few outlying data points. These genes have not previously been identified as associated with renal tumors. Adrenomedullin was recently associated with angiogenesis (Nikitenko, L. L. et al., Mol. Hum. Reprod. 6:811-819 (2000), although it has been recognized as a protein secreted by endothelial and other cell types. There are reports indicating that the chemokine receptor, CXCR4, to angiogenesis. Endothelial- and tumor cell-associated expression of CXCR4 and its ligand SDF-1 has been described in glioblastomas (Rempel, S. A. et al., Clin. Cancer Res. 6:102-111 (2000)) and pancreatic tumors (Koshiba, T. et al., Clin. Cancer Res. 6:3530-3535 (2000)), but not in renal cell carcinomas. Many of the genes exhibiting an elevated median tumor:normal ratio in renal tumor versus normal tissues were matrix or matrix-associated molecules, including several collagens and laminins. This may relate to the invasion of renal cancer cells into normal tissue and alterations in extracellular matrix composition that may accompany this process. Several endothelial marker genes, including CD31 (platelet/endothelial cell adhesion molecule, PECAM) and VE-cadherin were also elevated in tumor versus normal tissue, consistant with the highly vascular nature of RCC.

It has been recognized that tumor tissue contains both pro- and anti-angiogenic factors. As shown in Table 3, several genes that have been associated with inhibition of angiogenesis, including TIMP-1 (tissue inhibitor of metalloproteinase (MMP)-1, an inhibitor of MMP2 and other MMPs) and thrombospondin-2 (TSP-2) (Hawighorst, T. eta l., EMBO J 20:2630-2640 (2001)) are disclosed herein as elevated in renal tumor tissue versus normal tissue. Thus, the methods of the invention include a method of limiting or preventing the growth of renal cell carcinoma by contacting a RCC with an agent that enhances the expression of TIMP-1 and/or TSP-2 in the RCC thereby inhibiting angiogenesis and decreasing the supply of nutrients to the tumor tissue.

Overexpression in Tumor:

The median Tumor:Normal expression ratio values for genes overexpressed in renal cell carcinoma are reported in Table 3. A median Tumor:Normal ratio >1.5 was typically used as the threshold value for amplification scoring. Table 3 indicates that significant amplification of the listed genes is associated with tumor such as renal cell carcinoma. Because amplification of these genes occurs in tumor, it is highly probable to play a significant role in tumor formation or growth. As a result, antagonists (e.g., antibodies, antisense nucleic acids) directed against one of these genes or its encoded protein would be expected to have utility in cancer therapy. Agonists of anti-angiogenic genes or the encoded protein, such as TSP-2 and TIMP1, would be expected to have utility in cancer therapy as well.

TABLE 3
Ratio of Median Intensity Values for Gene Expression
in Renal Cell Carcinoma versus Normal Renal Tissue.

| | | | Median | |
|---|---|---|---|---|
| | Accession | Median | Intensity | Median |
| Gene | Number | Intensity | Normal | Tumor: |
| Genes Upregulated in | | Renal | Renal | Normal |
| Renal Tumors | | Tumors | Tissue | Ratio |
| CXCR4 | L06797 | 522.6 | 79.6 | 6.6 |
| Laminin alpha 4 | S78569 | 114.9 | 21.6 | 5.3 |
| TIMP1 | D11139 | 1224.9 | 235.4 | 5.2 |
| Type IV collagen alpha 1 | M26576 | 989.3 | 279.4 | 3.5 |
| Laminin alpha 3 (nicein) | L34155 | 23.4 | 6.9 | 3.4 |
| Adrenomedullin | D14874 | 825.3 | 244.8 | 3.4 |
| Type VI collagen alpha 2 | X15882 | 481.6 | 143.1 | 3.4 |
| Thrombospondin 2 | L12350 | 60 | 17.9 | 3.4 |
| Type I collagen alpha 2 | V00503 | 309.7 | 95.4 | 3.2 |
| Type VI collagen alpha 3 | X52022 | 250.1 | 86.3 | 2.9 |
| Latent TGFbeta binding | Z37976 | 84.8 | 31.1 | 2.7 |
| protein 2 | | | | |
| Serine or cystein | D83174 | 584.4 | 218.2 | 2.7 |
| protease inhibitor heat | | | | |
| shock protein 47 (HSP47) | | | | |
| Procollagen-lysine, | U84573 | 324.5 | 126.4 | 2.6 |
| 2-oxoglutarate | | | | |
| 5-dioxygenase | | | | |
| Connexin 43 | X52947 | 149.4 | 64.6 | 2.3 |
| Type IV collagen alpha 2 | X05610 | 1191.8 | 534.9 | 2.2 |
| Connexin 37 | M96789 | 101.2 | 45.5 | 2.2 |
| Ephrin A1 | M57730 | 279.1 | 125.8 | 2.2 |
| Laminin Beta 2 | M55210 | 224.9 | 107.2 | 2.1 |
| IntegrinAlpha 1 | X68742 | 204.2 | 100.2 | 2.0 |
| Hevin | X86693 | 988.6 | 559.3 | 1.8 |
| Stanniocalcin 1 | U25997 | 262.6 | 158.6 | 1.7 |
| Thrombospondin 4 | Z19585 | 5.9 | 3.6 | 1.6 |
| CD36 | M98399 | 5.5 | 3.4 | 1.6 |

The genes listed in Table 3 are uniquely disclosed herein as overexressed in renal cell carcinoma. Thus, according to the invention, determining the overexpression of any one or a plurality of these genes in renal tissue suspected of being cancerous is useful in the diagnosis of renal cell carcinoma. In addition, according to the invention, a method of antagonizing the overexpression of these genes is useful in treating renal cell carcinoma.

The following examples provide guidance for the preparation of the polypeptides encoded by the genes overexpressed in renal cell carcinoma, which polypeptides are useful in preparation of antagonist or agonist antibodies or small molecules capable of modulating their function.

### Example 2

Expression of CXCR4; Laminin Alpha 4; TIMP1; Type IV Collagen Alpha 1; Laminin Alpha 3; Adrenomedullin; Thrombospondin 2; Type I Collagen Alpha 2; Type VI Collagen Alpha 2; Type VI Collagen Alpha 3; Latent TGFbeta Binding Protein 2 (LTBP2); Serine or Cystein Protease Inhibitor Heat Shock Protein (HSP47); Procollagen-Lysine, 2-Oxoglutarate 5-Dioxygenase; Connexin 43; Type IV Collagen Alpha 2; Connexin 37; Ephrin A1; Laminin Beta 2; Integrin Alpha 1; Stanniocalcin 1; Thrombospondin 4; or CD36 Polypeptides in E. coli.

This example illustrates preparation of an unglycosylated form of CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 by recombinant expression in E. coli.

The DNA sequence encoding the polypeptide of interest is initially amplified using selected PCR primers. The primers should contain restriction enzyme sites which correspond to the restriction enzyme sites on the selected expression vector. A variety of expression vectors may be employed. An example of a suitable vector is pBR322 (derived from E. coli; see Bolivar et al., Gene, 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a poly-His leader (including the first six STII codons, poly-His sequence, and enterokinase cleavage site), the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 coding region, lambda transcriptional terminator, and an argu gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 protein can then be purified using a metal chelating column under conditions that allow tight binding of the protein.

A polypeptide is expressed in E. coli in a poly-His tagged form using the following procedure. The DNA encoding the selected polypeptide is initially amplified using selected PCR primers. The primers preferably contain restriction enzyme sites which correspond to the restriction enzyme sites on the selected expression vector, and other useful sequences providing for efficient and reliable translation initiation, rapid purification on a metal chelation column, and proteolytic removal with enterokinase. The PCR-amplified, poly-His tagged sequences are then ligated into an expression vector, which is used to transform an E. coli host based on strain 52 (W3110 fuhA(tonA) lon galE rpoHts(htpRts) clpP(lacIq). Transformants were first grown in LB containing 50 mg/ml carbenicillin at 30° C. with shaking until an O.D. of 3-5 at 600 nm is reached. Cultures are then diluted 50-100 fold into CRAP media (prepared by mixing 3.57 g (NH4)2SO4, 0.71 g sodium citrate•2H₂O, 1.07 g KCl, 5.36 g Difco yeast extract, 5.36 g Sheffield hycase SF in 500 ml water, as well as 110 mM MPOS, pH 7.3, 0.55% (w/v) glucose and 7 mM MgSO₄) and grown for approximately 20-30 hours at 30° C. with shaking. Samples are removed to verify expression by SDS-PAGE analysis, and the bulk culture is centrifuged to pellet the cells. Cell pellets are frozen until purification and refolding.

E. coli paste from 0.5 to 1 L fermentations (6-10 g pellets) is resuspended in 10 volumes (w/v) in 7 M guanidine, 20 mM Tris, pH 8 buffer. Solid sodium sulfite and sodium tetrathionate were added to make final concentrations of 0. 1M and 0.02 M, respectively, and the solution is stirred overnight at 4° C. This step results in a denatured protein with all cysteine residues blocked by sulfitolization. The solution is centrifuged at 40,000 rpm in a Beckman Ultracentifuge for 30 min. The supernatant is diluted with 3-5 volumes of metal chelate column buffer (6 M guanidine, 20 mM Tris, pH 7.4) and filtered through 0.22 micron filters to clarify. The clarified extract is loaded onto a 5 ml Qiagen Ni²⁺-NTA metal chelate column equilibrated in the metal chelate column buffer. The column is washed with additional buffer containing 50 mM imidazole (Calbiochem, Utrol grade), pH 7.4. The proteins were eluted with buffer containing 250 mM imidazole. Fractions containing the desired protein are pooled and stored at 4° C. Protein concentration is estimated by its absorbance at 280 nm using the calculated extinction coefficient based on its amino acid sequence.

The proteins are refolded by diluting sample slowly into freshly prepared refolding buffer consisting of: 20 mM Tris, pH 8.6, 0.3 M NaCl, 2.5 M urea, 5 mM cysteine, 20 mM glycine and 1 mM EDTA. Refolding volumes arechosen so that the final protein concentration is between 50 to 100 micrograms/ml. The refolding solution is stirred gently at 4° C. for 12-36 hours. The refolding reaction is quenched by the addition of TFA to a final concentration of 0.4% (pH of approximately 3). Before further purification of the protein, the solution is filtered through a 0.22 micron filter and acetonitrile is added to 2-10% final concentration. The refolded protein is chromatographed on a Poros R1/H reversed phase column using a mobile buffer of 0.1% TFA with elution with a gradient of acetonitrile from 10 to 80%. Aliquots of fractions with A₂₈₀ absorbance analyzed on SDS polyacrylamide gels and fractions containing homogeneous refolded protein are pooled. Generally, the properly refolded species of most proteins are eluted at the lowest concentrations of acetonitrile since those species are the most compact with their hydrophobic interiors shielded from interaction with the reversed phase resin. Aggregated species are usually eluted at higher acetonitrile concentrations. In addition to resolving misfolded forms of proteins from the desired form, the reversed phase step also removes endotoxin from the samples.

Fractions containing the desired folded PRO1788 and PRO1555 proteins are pooled and the acetonitrile removed using a gentle stream of nitrogen directed at the solution. Proteins are formulated into 20 mM Hepes, pH 6.8 with 0.14 M sodium chloride and 4% mannitol by dialysis or by gel filtration using G25 Superfine (Pharmacia) resins equilibrated in the formulation buffer and sterile filtered.

### Example 3

Expression of CXCR4; Laminin Alpha 4; TIMP1; Type IV Collagen Alpha 1; Laminin Alpha 3; Adrenomedullin; Thrombospondin 2; Type I Collagen Alpha 2; Type VI Collagen Alpha 2; Type VI Collagen Alpha 3; Latent TGFbeta Binding Protein 2 (LTBP2); Serine or Cystein Protease Inhibitor Heat Shock Protein (HSP47); Procollagen-Lysine, 2-Oxoglutarate 5-Dioxygenase; Connexin 43; Type IV Collagen Alpha 2; Connexin 37; Ephrin A1; Laminin Beta 2; Integrin Alpha 1; Stanniocalcin 1; Thrombospondin 4; or CD36 in Mammalian Cells.

This example illustrates preparation of a potentially glycosylated form of CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 by recombinant expression in mammalian cells.

The vector, pRK5 (see EP 307,247, published March 15, 1989), is employed as the expression vector. Optionally, the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 encoding DNA is ligated into pRK5 with selected restriction enzymes to allow insertion of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 encoding DNA using ligation methods such as described in Sambrook et al., supra. The resulting vector is called pRK5-CXCR4; pRK5-Laminin alpha 4; pRK5-TIMP1; pRK5-Type IV collagen alpha 1; pRK5-Laminin alpha 3; pRK5-Adrenomedullin; pRK5-Thrombospondin 2; pRK5-Type I collagen alpha 2; pRK5-Type VI collagen alpha 2; pRK5-Type VI collagen alpha 3; pRK5-Latent TGFbeta binding protein 2 (pRK5-LTBP2); pRK5-Serine or cystein protease inhibitor heat shock protein (pRK5-HSP47); pRK5-Procollagen-lysine, 2-oxoglutarate 5-dioxygenase; pRK5-connexin 43; pRK5-Type IV collagen alpha 2; pRK5-Connexin 37; pRK5-Ephrin A1; pRK5-Laminin beta 2; pRK5-Integrin alpha 1; pRK5-Stanniocalcin 1; pRK5-Thrombospondin 4; or pRK5-CD36.

In one embodiment, the selected host cells may be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-CXCR4; pRK5-Laminin alpha 4; pRK5-TIMP1; pRK5-Type IV collagen alpha 1; pRK5-Laminin alpha 3; pRK5-Adrenomedullin; pRK5-Thrombospondin 2; pRK5-Type I collagen alpha 2; pRK5-Type VI collagen alpha 2; pRK5-Type VI collagen alpha 3; pRK5-Latent TGFbeta binding protein 2 (pRK5-LTBP2); pRK5-Serine or cystein protease inhibitor heat shock protein (pRK5-HSP47); pRK5-Procollagen-lysine, 2-oxoglutarate 5-dioxygenase; pRK5-connexin 43; pRK5-Type IV collagen alpha 2; pRK5-Connexin 37; pRK5-Ephrin A1; pRK5-Laminin beta 2; pRK5-Integrin alpha 1; pRK5-Stanniocalcin 1; pRK5-Thrombospondin 4; or pRK5-CD36 DNA is mixed with about 1 μg DNA encoding the VA RNA gene [Thimmappaya et al., Cell, 31:543 (1982)] and dissolved in 500 μl of 1 mM Tris-HCl, 0.1 mM EDTA, 0.227 M CaCl₂. To this mixture is added, dropwise, 500 μl of 50 mM HEPES (pH 7.35), 280 mM NaCl, 1.5 mM NAPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml ³⁵S-cysteine and 200 μCi/ml ³⁵S-methionine. After a 12 hour incubation, the conditioned medium is collected, concentrated on a spin filter, and loaded onto a 15% SDS gel. The processed gel may be dried and exposed to film for a selected period of time to reveal the presence of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide. The cultures containing transfected cells may undergo further incubation (in serum free medium) and the medium is tested in selected bioassays.

In an alternative technique, CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 encoding DNA may be introduced into 293 cells transiently using the dextran sulfate method described by Somparyrac et al., Proc. Natl. Acad. Sci., 12:7575 (1981). 293 cells are grown to maximal density in a spinner flask and 700 μg pRK5-CXCR4; pRK5-Laminin alpha 4; pRK5-TIMP1; pRK5-Type IV collagen alpha 1; pRK5-Laminin alpha 3; pRK5-Adrenomedullin; pRK5-Thrombospondin 2; pRK5-Type I collagen alpha 2; pRK5-Type VI collagen alpha 2; pRK5-Type VI collagen alpha 3; pRK5-Latent TGFbeta binding protein 2 (pRK5-LTBP2); pRK5-Serine or cystein protease inhibitor heat shock protein (pRK5-HSP47); pRK5-Procollagen-lysine, 2-oxoglutarate 5-dioxygenase; pRK5-connexin 43; pRK5-Type IV collagen alpha 2; pRK5-Connexin 37; pRK5-Ephrin A1; pRK5-Laminin beta 2; pRK5-Integrin alpha 1; pRK5-Stanniocalcin 1; pRK5-Thrombospondin 4; or pRK5-CD36 DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0.1 μg/ml bovine transferrin. After about four days, the conditioned media is centrifuged and filtered to remove cells and debris. The sample containing expressed CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 can then be concentrated and purified by any selected method, such as dialysis and/or column chromatography.

In another embodiment CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 can be expressed in CHO cells. The pRK5-CXCR4; pRK5-Laminin alpha 4; pRK5-TIMP1; pRK5-Type IV collagen alpha 1; pRK5-Laminin alpha 3; pRK5-Adrenomedullin; pRK5-Thrombospondin 2; pRK5-Type I collagen alpha 2; pRK5-Type VI collagen alpha 2; pRK5-Type VI collagen alpha 3; pRK5-Latent TGFbeta binding protein 2 (pRK5-LTBP2); pRK5-Serine or cystein protease inhibitor heat shock protein (pRK5-HSP47); pRK5-Procollagen-lysine, 2-oxoglutarate 5-dioxygenase; pRK5-connexin 43; pRK5-Type IV collagen alpha 2; pRK5-Connexin 37; pRK5-Ephrin A1; pRK5-Laminin beta 2; pRK5-Integrin alpha 1; pRK5-Stanniocalcin 1; pRK5-Thrombospondin 4; or pRK5-CD36 vector can be transfected into CHO cells using known reagents such as CaPO₄ or DEAE-dextran. As described above, the cell cultures can be incubated, and the medium replaced with culture medium (alone) or medium containing a radiolabel such as ³⁵S-methionine. After determining the presence of the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, the culture medium may be replaced with serum free medium. Preferably, the cultures are incubated for about 6 days, and then the conditioned medium is harvested. The medium containing the expressed CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 can then be concentrated and purified by any selected method.

Epitope-tagged CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 may also be expressed in host CHO cells. The CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 may be subcloned out of the pRK5 vector. The subclone insert can undergo PCR to fuse in frame with a selected epitope tag such as a poly-His tag into a Baculovirus expression vector. The poly-His tagged CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 insert can then be subcloned into a SV40 driven vector containing a selection marker such as DHFR for selection of stable clones. Finally, the CHO cells can be transfected (as described above) with the SV40 driven vector. Labeling may be performed, as described above, to verify expression. The culture medium containing the expressed poly-His tagged CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 can then be concentrated and purified by any selected method, such as by Ni²⁺-chelate affinity chromatography. Expression in CHO and/or COS cells may also be accomplished by a transient expression procedure.

CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 can be expressed in CHO cells by a stable expression procedure. For example, stable expression in CHO cells is performed using the following procedure. The proteins are expressed as an IgG construct (immunoadhesin), in which the coding sequences for the soluble forms (e.g., extracellular domains) of the respective proteins are fused to an IgG1 constant region sequence containing the hinge, CH2 and CH2 domains and/or in a poly-His tagged form.

Following PCR amplification, the respective DNAs are subcloned in a CHO expression vector using standard techniques as described in Ausubel et al., Current Protocols of Molecular Biology, Unit 3.16, John Wiley and Sons (1997). CHO expression vectors are constructed to have compatible restriction sites 5′ and 3′ of the DNA of interest to allow the convenient shuttling of cDNA's. The vector used for expression in CHO cells is as described in Lucas et al., Nucl. Acids Res., 24:9 (1774-1779 (1996), and uses the SV40 early promoter/enhancer to drive expression of the cDNA of interest and dihydrofolate reductase (DHFR). DHFR expression permits selection for stable maintenance of the plasmid following transfection.

Twelve micrograms of the desired plasmid DNA are introduced into approximately 10 million CHO cells using commercially available transfection reagents Superfect® (Quiagen), Dosper® or Fugene® (Boehringer Mannheim). The cells are grown as described in Lucas et al., supra. Approximately 3×10⁻⁷ cells are frozen in an ampule for further growth and production as described below.

The ampules containing the plasmid DNA are thawed by placement into a water bath and mixted by vortexing. The contents are pipetted into a centrifuge tube containing 10 mls of media and centrifuged at 1000 rpm for 5 minutes. The supernatant is aspirated and the cells are resuspended in 10 ml of selective media (0.2 μm filtered PS20 with 5% 0.2 μm diafiltered fetal bovine serum). The cells are then aliquoted into a 100 ml spinner containing 90 ml of selective media. After 1-2 days, the cells are transferred into a 250 ml spinner filled with 150 ml selective growth medium and incubated at 37° C. After another 2-3 days, 250 ml, 500 ml and 2000 ml spinners are seeded with 3×10⁵ cells/ml. The cell media is exchanged with fresh media by centrifugation and resuspension in production medium. Although any suitable CHO media may be employed, a production medium described in U.S. Pat. No. 5,122,469, issued Jun. 16, 1992 may be used. 3 L production spinner is seeded at 1.2×10⁶ cells/ml. On day 0, the cell number and pH are determined. On day 1, the spinner is sampled and sparging with filtered air is commenced. On day 2, the spinner is sampled, the temperature shifted to 33° C., and 30 ml of 500 g/L glucose and 0.6 ml of 10% antifoam (e.g., 35% polydimethylsiloxane emulsion, Dow Corning 365 Medical Grade Emulsion) added. Throughout the production, the pH is adjusted as necessary to keep at around 7.2. After 10 days, or until viability dropped below 70%, the cell culture is harvested by centrifugation and filtered through a 0.22 μm filter. The filtrate is either stored at 4° C. or immediately loaded onto columns for purification.

For the poly-His tagged constructs, the proteins are purified using a Ni²⁺-NTA column (Qiagen). Before purification, imidazole is added to the conditioned media to a concentration of 5 mM. The conditioned media is pumped onto a 6 ml Ni²⁺-NTA column equilibrated in 20 mM Hepes, pH 7.4, buffer containing 0.3 M NaCl and 5 mM imidazole at a flow rate of 4-5 ml/min. at 4° C. After loading, the column is washed with additional equilibration buffer and the protein eluted with equilibration buffer containing 0.25 M imidazole. The highly purified protein is subsequently desalted into a storage buffer containing 10 mM Hepes, 0.14 M NaCl and 4% mannitol, pH 6.8, with a 25 ml G25 Superfine (Pharmacia) column and stored at −80° C.

Immunoadhesin (Fc containing) constructs are purified from the conditioned media as follows. The conditioned medium is pumped onto a 5 ml Protein A column (Pharmacia) which has been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 μl of 1 M Tris buffer, pH 9. The highly purified protein is subsequently desalted into storage buffer as described above for the poly-His tagged proteins. The homogeneity is assessed by SDS polyacrylamide gels and by N-terminal amino acid sequencing by Edman degradation.

### Example 4

Expression of CXCR4; Laminin Alpha 4; TIMP1; Type IV Collagen Alpha 1; Laminin Alpha 3; Adrenomedullin; Thrombospondin 2; Type I Collagen Alpha 2; Type VI Collagen Alpha 2; Type VI Collagen Alpha 3; Latent TGFbeta Binding Protein 2 (LTBP2); Serine or Cystein Protease Inhibitor Heat Shock Protein (HSP47); Procollagen-Lysine, 2-Oxoglutarate 5-Dioxygenase; Connexin 43; Type IV Collagen Alpha 2; Connexin 37; Ephrin A1; Laminin Beta 2; Integrin Alpha 1; Stanniocalcin 1; Thrombospondin 4; or CD36 Polypeptides in Yeast

The following method describes recombinant expression of CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 in yeast.

First, yeast expression vectors are constructed for intracellular production or secretion of CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 from the ADH2/GAPDH promoter. DNA encoding CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 and the promoter is inserted into suitable restriction enzyme sites in the selected plasmid to direct intracellular expression of CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36. For secretion, DNA encoding CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 can be cloned into the selected plasmid, together with DNA encoding the ADH2/GAPDH promoter, a native CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 signal peptide (if applicable) or other mammalian signal peptide, or, for example, a yeast alpha-factor or invertase secretory signal/leader sequence, and linker sequences (if needed) for expression of CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36.

Yeast cells, such as yeast strain AB110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 can subsequently be isolated and purified by removing the yeast cells from the fermentation medium by centrifugation and then concentrating the medium using selected cartridge filters. The concentrate containing CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 may further be purified using selected column chromatography resins.

### Example 5

Expression of CXCR4; Laminin Alpha 4; TIMP1; Type IV Collagen Alpha 1; Laminin Alpha 3; Adrenomedullin; Thrombospondin 2; Type I Collagen Alpha 2; Type VI Collagen Alpha 2; Type VI Collagen Alpha 3; Latent TGFbeta Binding Protein 2 (LTBP2); Serine or Cystein Protease Inhibitor Heat Shock Protein (HSP47); Procollagen-Lysine, 2-Oxoglutarate 5-Dioxygenase; Connexin 43; Type IV Collagen Alpha 2; Connexin 37; Ephrin A1; Laminin Beta 2; Integrin Alpha 1; Stanniocalcin 1; Thrombospondin 4; or CD36 Polypeptides in Baculovirus-Infected Insect Cells

The following method describes recombinant expression in Baculovirus-infected insect cells.

The sequence coding for CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 is fused upstream of an epitope tag contained within a baculovirus expression vector. Such epitope tags include poly-His tags and immunoglobulin tags (like Fc regions of IgG). A variety of plasmids may be employed, including plasmids derived from commercially available plasmids such as pVL1393 (Novagen). Briefly, the sequence encoding CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 or the desired portion of the coding sequence of CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 (such as the sequence encoding the extracellular domain of a transmembrane protein or the sequence encoding the mature protein if the protein is extracellular) is amplified by PCR with primers complementary to the 5′ and 3′ regions. The 5′ primer may incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4-5 days of incubation at 28° C., the released viruses are harvested and used for further amplifications. Viral infection and protein expression are performed as described by O'Reilley et al., Baculovirus expression vectors: A Laboratory Manual, Oxford: Oxford University Press (1994).

Expressed poly-His tagged CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 can then be purified, for example, by Ni²⁺-chelate affinity chromatography as follows. Extracts are prepared from recombinant virus-infected Sf9 cells as described by Rupert et al., Nature, 362:175-179 (1993). Briefly, Sf9 cells are washed, resuspended in sonication buffer (25 ml Hepes, pH 7.9; 12.5 mM MgCl₂; 0.1 mM EDTA; 10% glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% glycerol, pH 7.8) and filtered through a 0.45 μm filter. A Ni²⁺-NTA agarose column (commercially available from Qiagen) is prepared with a bed volume of 5 ml, washed with 25 ml of water and equilibrated with 25 ml of loading buffer. The filtered cell extract is loaded onto the column at 0.5 ml per minute. The column is washed to baseline A₂₈₀ with loading buffer, at which point fraction collection is started. Next, the column is washed with a secondary wash buffer (50 mM phosphate; 300 mM NaCl, 10% glycerol, pH 6.0), which elutes nonspecifically bound protein. After reaching A₂₈₀ baseline again, the column is developed with a 0 to 500 mM imidazole gradient in the secondary wash buffer. One ml fractions are collected and analyzed by SDS-PAGE and silver staining or Western blot with Ni²⁺-NTA-conjugated to alkaline phosphatase (Qiagen). Fractions containing the eluted His₁₀-tagged CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36, respectively, are pooled and dialyzed against loading buffer.

Alternatively, purification of the IgG tagged (or Fc tagged) CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 can be performed using known chromatography techniques, including for instance, Protein A or protein G column chromatography.

While expression is actually performed in a 0.5-2 L scale, it can be readily scaled up for larger (e.g., 8 L) preparations. The proteins are expressed as an IgG construct (immunoadhesin), in which the protein extracellular region is fused to an IgG1 constant region sequence containing the hinge, CH2 and CH3 domains and/or in poly-His tagged forms.

Following PCR amplification, the respective coding sequences are subcloned into a baculovirus expression vector (pb.PH.IgG for IgG fusions and pb.PH.His.c for poly-His tagged proteins), and the vector and Baculogold® baculovirus DNA (Pharmingen) are co-transfected into 105 Spodoptera frugiperda (“Sf9”) cells (ATCC CRL 1711), using Lipofectin (Gibco BRL). pb.PH.IgG and pb.PH.His are modifications of the commercially available baculovirus expression vector pVL1393 (Pharmingen), with modified polylinker regions to include the His or Fc tag sequences. The cells are grown in Hink's TNM-FH medium supplemented with 10% FBS (Hyclone). Cells are incubated for 5 days at 28° C. The supernatant is harvested and subsequently used for the first viral amplification by infecting Sf9 cells in Hink's TNM-FH medium supplemented with 10% FBS at an approximate multiplicity of infection (MOI) of 10. Cells are incubated for 3 days at 28° C. The supernatant is harvested and the expression of the constructs in the baculovirus expression vector is determined by batch binding of 1 ml of supernatant to 25 ml of Ni²⁺-NTA beads (QIAGEN) for histidine tagged proteins or Protein-A Sepharose CL-4B beads (Pharmacia) for IgG tagged proteins followed by SDS-PAGE analysis comparing to a known concentration of protein standard by Coomassie blue staining.

The first viral amplification supernatant is used to infect a spinner culture (500 ml) of Sf9 cells grown in ESF-921 medium (Expression Systems LLC) at an approximate MOI of 0.1. Cells are incubated for 3 days at 28° C. The supernatant is harvested and filtered. Batch binding and SDS-PAGE analysis are repeated, as necessary, until expression of the spinner culture is confirmed.

The conditioned medium from the transfected cells (0.5 to 3 L) is harvested by centrifugation to remove the cells and filtered through 0.22 micron filters. For the poly-His tagged constructs, the protein construct is purified using a Ni²⁺-NTA column (Qiagen). Before purification, imidazole is added to the conditioned media to a concentration of 5 mM. The conditioned media is pumped onto a 6 ml Ni²⁺-NTA column equilibrated in 20 mM Hepes, pH 7.4, buffer containing 0.3 M NaCl and 5 mM imidazole at a flow rate of 4-5 ml/min. at 4° C. After loading, the column is washed with additional equilibration buffer and the protein eluted with equilibration buffer containing 0.25 M imidazole. The highly purified protein is subsequently desalted into a storage buffer containing 10 mM Hepes, 0.14 M NaCl and 4% mannitol, pH 6.8, with a 25 ml G25 Superfine (Pharmacia) column and stored at −80° C.

Immunoadhesin (Fc containing) constructs of proteins are purified from the conditioned media as follows. The conditioned media is pumped onto a 5 ml Protein A column (Pharmacia) which has been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 ml of 1 M Tris buffer, pH 9. The highly purified protein is subsequently desalted into storage buffer as described above for the poly-His tagged proteins. The homogeneity of the proteins is verified by SDS polyacrylamide gel (PEG) electrophoresis and N-terminal amino acid sequencing by Edman degradation.

Alternatively, a modified baculovirus procedure may be used incorporating high 5 cells. In this procedure, the DNA encoding the desired sequence is amplified with suitable systems, such as Pfu (Stratagene), or fused upstream (5′-of) of an epitope tag contained with a baculovirus expression vector. Such epitope tags include poly-His tags and immunoglobulin tags (like Fc regions of IgG). A variety of plasmids may be employed, including plasmids derived from commercially available plasmids such as pIE1-1 (Novagen). The pIE1-1 and pIE1-2 vectors are designed for constitutive expression of recombinant proteins from the baculovirus ie1 promoter in stably-transformed insect cells. The plasmids differ only in the orientation of the multiple cloning sites and contain all promoter sequences known to be important for ie1-mediated gene expression in uninfected insect cells as well as the hr5 enhancer element. pIE1-1 and pIE1-2 include the translation initiation site and can be used to produce fusion proteins. Briefly, the desired sequence or the desired portion of the sequence (such as the sequence encoding the extracellular domain of a transmembrane protein) is amplified by PCR with primers complementary to the 5′ and 3′ regions. The 5′ primer may incorporate flanking (selected) restriction enzyme sites. The product is then digested with those selected restriction enzymes and subcloned into the expression vector. For example, derivatives of pIE1-1 can include the Fc region of human IgG (pb.PH.IgG) or an 8 histidine (pb.PH.His) tag downstream (3′-of) the desired sequence. Preferably, the vector construct is sequenced for confirmation.

High 5 cells are grown to a confluency of 50% under the conditions of 27° C., no CO₂, NO pen/strep. For each 150 mm plate, 30 μg of pIE based vector containing the sequence is mixed with 1 ml Ex-Cell medium (Media: Ex-Cell 401+1/100 L-Glu JRH Biosciences #14401-78P (note: this media is light sensitive)), and in a separate tube, 100 μl of CellFectin (CellFECTIN (GibcoBRL #10362-010) (vortexed to mix)) is mixed with 1 ml of Ex-Cell medium. The two solutions are combined and allowed to incubate at room temperature for 15 minutes. 8 ml of Ex-Cell media is added to the 2 ml of DNA/CellFECTIN mix and this is layered on high 5 cells that has been washed once with Ex-Cell media. The plate is then incubated in darkness for 1 hour at room temperature. The DNA/CellFECTIN mix is then aspirated, and the cells are washed once with Ex-Cell to remove excess CellFECTIN, 30 ml of fresh Ex-Cell media is added and the cells are incubated for 3 days at 28° C. The supernatant is harvested and the expression of the sequence in the baculovirus expression vector is determined by batch binding of 1 ml of supernatant to 25 ml of Ni²⁺-NTA beads (QIAGEN) for bistidine tagged proteins or Protein-A Sepharose CL-4B beads (Pharmacia) for IgG tagged proteins followed by SDS-PAGE analysis comparing to a known concentration of protein standard by Coomassie blue staining.

The conditioned media from the transfected cells (0.5 to 3 L) is harvested by centrifugation to remove the cells and filtered through 0.22 micron filters. For the poly-His tagged constructs, the protein comprising the sequence is purified using a Ni²⁺-NTA column (Qiagen). Before purification, imidazole is added to the conditioned media to a concentration of 5 mM. The conditioned media is pumped onto a 6 ml Ni²⁺-NTA column equilibrated in 20 mM Hepes, pH 7.4, buffer containing 0.3 M NaCl and 5 mM imidazole at a flow rate of 4-5 ml/min. at 48° C. After loading, the column is washed with additional equilibration buffer and the protein eluted with equilibration buffer containing 0.25 M imidazole. The highly purified protein is then subsequently desalted into a storage buffer containing 10 mM Hepes, 0.14 M NaCl and 4% mannitol, pH 6.8, with a 25 ml G25 Superfine (Pharmacia) column and stored at −80° C.

Immunoadhesin (Fc containing) constructs of proteins are purified from the conditioned media as follows. The conditioned media is pumped onto a 5 ml Protein A column (Pharmacia) which has been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 ml of 1 M Tris buffer, pH 9. The highly purified protein is subsequently desalted into storage buffer as described above for the poly-His tagged proteins. The homogeneity of the sequence is assessed by SDS polyacrylamide gels and by N-terminal amino acid sequencing by Edman degradation and other analytical procedures as desired or necessary.

### Example 6

Preparation of Antibodies that Bind CXCR4; Laminin Alpha 4; TIMP1; Type IV Collagen Alpha 1; Laminin Alpha 3; Adrenomedullin; Thrombospondin 2; Type I Collagen Alpha 2; Type VI Collagen Alpha 2; Type VI Collagen Alpha 3; Latent TGFbeta Binding Protein 2 (LTBP2); Serine or Cystein Protease Inhibitor Heat Shock Protein (HSP47); Procollagen-Lysine, 2-Oxoglutarate 5-Dioxygenase; Connexin 43; Type IV Collagen Alpha 2; Connexin 37; Ephrin A1; Laminin Beta 2; Integrin Alpha 1; Stanniocalcin 1; Thrombospondin 4; or CD36

This example illustrates preparation of monoclonal antibodies which can specifically bind CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36.

Techniques for producing the monoclonal antibodies are known in the art and are described, for instance, in Goding, supra. Immunogens that may be employed include purified CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 fusion proteins containing CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 and cells expressing recombinant CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 on the cell surface. Selection of the immunogen can be made by the skilled artisan without undue experimentation.

Mice, such as Balb/c, are immunized with the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 immunogen emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine orcysteinprotease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibodies.

After a suitable antibody titer has been detected, the animals “positive” for antibodies can be injected with a final intravenous injection of CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36. Determination of “positive” hybridoma cells secreting the desired monoclonal antibodies against CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 is within the skill in the art.

The positive hybridoma cells can be injected intraperitoneally into syngeneic Balb/c mice to produce ascites containing the anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

What is claimed is:

1. An isolated antibody that binds to a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide.

2. The antibody of claim 1 which specifically binds to said polypeptide.

3. The antibody of claim 1 which induces the death of a cell that expresses said polypeptide.

4. The antibody of claim 3, wherein said cell is part of a renal cell carcinoma and the cell overexpresses said polypeptide as compared to a normal cell of the same tissue type.

5. The antibody of claim 1 which is a monoclonal antibody.

6. The antibody of claim 5 which comprises a non-human complementarity determining region (CDR) or a human framework region (FR).

7. The antibody of claim 1 which is labeled.

8. The antibody of claim 1 which is an antibody fragment or a single-chain antibody.

9. A composition of matter which comprises an antibody of claim 1 in admixture with a pharmaceutically acceptable carrier.

10. The composition of matter of claim 9 which comprises a therapeutically effective amount of said antibody.

11. The composition of matter of claim 9 which further comprises a cytotoxic or a chemotherapeutic agent.

12. A method for producing an antibody that binds to a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, said method comprising culturing a host cell comprising nucleic acid encoding the antibody under conditions sufficient to allow expression of said antibody and recovering said antibody from the cell culture.

13. An antagonist of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide.

14. The antagonist of claim 13, wherein said antagonist inhibits growth of a renal cell carcinoma.

15. A method of diagnosing tumor in a mammal, said method comprising detecting the level of expression of a gene encoding a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide (a) in a test sample of tissue cells obtained from the mammal, and (b) in a control sample of known normal tissue cells of the same cell type, wherein a higher expression level in the test sample, as compared to the control sample, is indicative of the presence of tumor in the mammal from which the test tissue cells are obtained.

16. The method of claim 15, wherein the test sample is from a renal cell carcinoma and the control sample is from normal renal tissue.

17. A method for determining the overexpression of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide (a) in a test tissue sample suspected of containing said polypeptide and (b) a control normal tissue sample of the same tissue type, said method comprising exposing the test and control tissue samples to an anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody and determining the relative binding of said antibody to said polypeptide in said samples.

18. The method of claim 17, wherein the test sample is from a renal cell carcinoma and the control sample is from normal renal tissue.

19. A method of diagnosing renal cell carcinoma in a mammal, said method comprising (a) contacting an anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody with a test sample of renal tissue cells obtained from the mammal suspected of having renal cell carcinoma and a control sample of normal renal tissue, and (b) detecting an increased formation of a complex between said antibody and a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide in the test sample relative to the normal sample, wherein the increased formation of a complex is indicative of the presence of renal cell carcinoma in said mammal.

20. The method of claim 17, 18, or 19, wherein said antibody is detectably labeled.

21. A renal cell carcinoma diagnostic kit comprising an anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody and a carrier in suitable packaging.

22. The kit of claim 21 which further comprises instructions for using said antibody to detect the increased presence of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide in a renal cell carcinoma tissue sample suspected of containing the same relative to a normal renal tissue sample.

23. The kit of claim 21 or 22, wherein said antibody is detectably labeled.

24. A method for inhibiting the growth of renal cell carcinoma, said method comprising exposing a renal cell carcinoma, in which a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide is overexpressed, to an effective amount of an agent that inhibits a biological activity of said polypeptide, wherein growth of said renal cell carcinoma is thereby inhibited.

25. The method of claim 24, wherein said agent is an anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine orcysteinprotease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody.

26. The method of claim 25, wherein said anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody induces cell death.

27. The method of claim 24, wherein the renal cell carcinoma is further exposed to radiation treatment, a cytotoxic agent or a chemotherapeutic agent.

28. A method for inhibiting the growth of a renal cell carcinoma, said method comprising exposing renal cell carcinoma tissue, in which a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide is overexpressed, to an effective amount of an agent that inhibits the expression of said polypeptide, wherein growth of said renal cell carcinoma is thereby inhibited.

29. The method of claim 28, wherein said polypeptide is overexpressed in renal cell carcinoma as compared to normal renal cells.

30. The method of claim 28, wherein said agent is an antisense oligonucleotide that hybridizes to a nucleic acid which encodes the CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide or the complement thereof.

31. The method of claim 30, wherein said renal cell carcinoma is further exposed to radiation treatment, a cytotoxic agent or a chemotherapeutic agent.

32. An article of manufacture, comprising:
a container;
a label on the container; and
a composition comprising an active agent contained within the container, wherein the composition is effective for inhibiting the growth of renal cell carcinoma and wherein the label on the container indicates that the composition is effective for treating conditions characterized by overexpression of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide in said renal cell carcinoma as compared to normal renal tissue.

33. The article of manufacture of claim 32, wherein said active agent inhibits a biological activity of and/or the expression of said CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide.

34. The article of manufacture of claim 33, wherein said active agent is an anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody.

35. The article of manufacture of claim 33, wherein said active agent is an antisense oligonucleotide.

36. A method of identifying a compound that inhibits an activity of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, said method comprising the steps of (a) contacting cells and a candidate compound to be screened in the presence of said polypeptide under conditions suitable for the induction of a cellular response normally induced by said polypeptide and (b) determining the induction of said cellular response to determine if the test compound is an effective antagonist, wherein the lack of induction of said cellular response is indicative of said compound being an effective antagonist.

37. The method of claim 36, wherein said candidate compound is an anti-CXCR4; anti-Laminin alpha 4; anti-TIMP1; anti-Type IV collagen alpha 1; anti-Laminin alpha 3; anti-Adrenomedullin; anti-Thrombospondin 2; anti-Type I collagen alpha 2; anti-Type VI collagen alpha 2; anti-Type VI collagen alpha 3; anti-Latent TGFbeta binding protein 2 (anti-LTBP2); anti-Serine or cystein protease inhibitor heat shock protein (anti-HSP47); anti-Procollagen-lysine, 2-oxoglutarate 5-dioxygenase; anti-connexin 43; anti-Type IV collagen alpha 2; anti-Connexin 37; anti-Ephrin A1; anti-Laminin beta 2; anti-Integrin alpha 1; anti-Stanniocalcin 1; anti-Thrombospondin 4; or anti-CD36 polypeptide antibody.

38. The method of claim 36, wherein a component, either said candidate compound or said CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, is immobilized on a solid support.

39. The method of claim 38, wherein a component, either said candidate compound or said CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide, is not immobilized on a solid support and is detectably labeled.

40. A method for identifying a compound that inhibits the expression of a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide in cells that express said polypeptide, wherein said method comprises contacting said cells with a candidate compound and determining whether expression of said polypeptide is inhibited.

41. The method of claim 40, wherein said candidate compound is an antisense oligonucleotide.
